SEQUENCE LISTING (37 sequences)

SEQ ID NO: 1 - DNA, 27 bp, Artificial Sequence
  primer
gagctcatcaccgcaggcgagtctctt

SEQ ID NO: 2 - DNA, 27 bp, Artificial Sequence
  primer
gagctcggtcttcgggactaatgcctt

SEQ ID NO: 3 - DNA, 26 bp, Artificial Sequence
  primer
ggggtaccggttttgttttgtgtgac

SEQ ID NO: 4 - DNA, 26 bp, Artificial Sequence
  primer
ggggtaccggtgtgtgatgatttcgc

SEQ ID NO: 5 - DNA, 21 bp, Artificial Sequence
  primer
tcgacaactccgagtttcagc

SEQ ID NO: 6 - DNA, 40 bp, Artificial Sequence
  primer
ctcctcgcggccgcatcgatccatagagcccaccgcatcc

SEQ ID NO: 7 - DNA, 43 bp, Artificial Sequence
  primer
ctcctcaagcttctcgagcacacggagcgcggctgccgacacg

SEQ ID NO: 8 - DNA, 35 bp, Artificial Sequence
  primer
ctcctcggtaccccatggaggccagcagagccagc

SEQ ID NO: 9 - DNA, 42 bp, Artificial Sequence
  primer
ctcctcgcggccgcactagttccgcgtgtccctttccgatgc

SEQ ID NO: 10 - DNA, 58 bp, Artificial Sequence
  primer
ctcctcctcgagaagcttatgcatgagctcgacgtctcggcggtaatgagatacgagc

SEQ ID NO: 11 - DNA, 32 bp, Artificial Sequence
  primer
ctcctcagaacccaggaccagggccacgttgg

SEQ ID NO: 12 - DNA, 33 bp, Artificial Sequence
  primer
ctcctcatggagacaaagcccaagacggcaacc

SEQ ID NO: 13 - DNA, 36 bp, Artificial Sequence
  primer
ctcctcggagaccggggttggggaatgaatccctcc

SEQ ID NO: 14 - DNA, 35 bp, Artificial Sequence
  primer
ctcctcgcggggcgtgggaggggctggggcggacc

SEQ ID NO: 15 - DNA, 20 bp, Artificial Sequence
  primer
ctgactggctttagcccctt

SEQ ID NO: 16 - DNA, 21 bp, Artificial Sequence
  primer
gccttagagagaggtgagagc

SEQ ID NO: 17 - DNA, 25 bp, Artificial Sequence
  primer
gccacaaaacactttcaggtacata

SEQ ID NO: 18 - DNA, 21 bp, Artificial Sequence
  primer
tgatgccttagagagaggtga

SEQ ID NO: 19 - DNA, 25 bp, Artificial Sequence
  primer
aagatttattagtgcgttttggtgc

SEQ ID NO: 20 - DNA, 21 bp, Artificial Sequence
  primer
cagaagcactggtagcaagga

SEQ ID NO: 21 - DNA, 1400 bp, Mus musculus
aggatctgacacgctgtccttagctaggcaccgtcagttacaccccaggtggacttaaccacccatgaagatgtgggggagcttttcagacctcatgcctcacctttctcatttacaaatgggtagactgatactattctcaaacttctatgaacattcacttacaacatataagccacaaaacactttcaggtacataaccaattcctgactggctttagccccttcccgcatcctgacccctgccatcatagtcgccaccaactcttagaaacaccgcaagcttctcaggtcaggttttccagattttcataactgcttcctcttccaaacccacacttatctcttctatggttcagcgtgaggtgaggtttgaatctgtcatgcctcaactaccccactccgctttgccacacgtgccagtgccgacgtaccccaccagtccatgcactttttataatggcatttgcaagctgcctattttcaactgctagcctcttatgcctgctgttcctcatttgaagaggaacaagcaggaagcccaggaaagtatacgccattcccttggtgcttctagcccacccacccctccatcctgcttagtgacagctgccccttctttaatcgatcagcgaggtgcatttgaaaacttattctagaaaacattggattttttttcctttgctctgtgtcaggagatgtgatttggtgaaccctggggtttgaagggacaagtctcagaaatggatgaggaaaggaaatccctcctttagggattcaagctcgactgagcacacctgttactcaaggaaccgacaataccctagtgagtgtgtgggcatgtgagcccatgagctggggattacagctcgacagtgctgggggttacgaaatcctatcaagagtcaccaagaagtcagtgtgggggtcctactttctcaaagtcacagacactctgaagagagatcctgtctgataaagaaagtgattaccacacgagccattcttgtctgcacagcaattctgagagcccatcctgggagctaggtgtgtagtgtttatcgtattgttgaggctcgtaaaaatcttgtatggctgcaggcgagccaaaccctttgcaggctttgcatctccggctgactctgaggaccaagcccaatttcttctcagtatataagggcacggcactgggctcaaggcagaggagttctcagctccttccatctcttgtctttgctctcacctctctctaaggcatcatgagtctacagtgtagctccaggtccctgtgccgcggtggcggtggcagcaggaacttcagctcaggctctgctggcctagtcagcttccagcgcagatccaccagcagctctatgcgccgcagcggtggaggtggtggt

SEQ ID NO: 22 - DNA, 1400 bp, Mus musculus
aaaaagaacattaggagaaaatgttccacaatctgcaagagagcgatttaaattcccccatgttgagctgagcagcaaccctttgcaacattgtataatgattcttctggaccggctatgctttcttctggttttaaacaattgtgtccatgtgggcaaatcattgatcattattttctgaagctgggaatttattctttctcttcatgatcacaagatttattagtgcgttttggtgcagttgggtgtgaatatgggaatgaagagaacgaacatgcactagctatttgtgtccaagtgcaaacacacacttctgttggacaacacttgtagactctcatgaacttcacattacgtcttctgctgggacagccctttcttctcagagagtgcgcatgagccaaggttcctttcctactactccctgataagtacagaaggtgagagtaatgctttaagaactcactattattgttactgtctctgccaggagaaaaattttatgctgaccaaataataatactaaatccaaattaaggcttgtgtttcacatacctgtggtctttatgtatttcttaaaagatagctactttttctttttacaaaaatattgtcggtgccagtttagaaatattagaatagaagaagacatatgggagtagttctcctctgagtctaggagacattcatactgggagatgctcacagatattttgttcacacacatgcatgcacacaccaaacaaacaaacaaacaaacaaaaaaccacaaaacaaaaaatggattttatgtacgaggacagccttgttacagcaagtgtctccactctcatccaagctgtggccccaaggtcactagacacttcatagttttatcctagatctctctggcacaccccaaaacaaacaactccatgctgcttcttaggaaaagatcaatttgagatttaaggagaaacactacaggagtttccaacactgagatcctgagagattgctatcctttggtctctccgtagtaagagatgaatgataaatgatcaagttgggggagtttgtctgcaatgccatttcagcaacacataggtatgaggcttgtaagcagatgctactggcaggcaaatactcccctcccagggttcggaaagtttccagcccagcaggtgtgtgtatatatagggactgagccagatcctttccaagagagtcgcagctccctcagtccctgctctctgcctgctttcagctgagtccttgctaccagtgcttctggttgccctagcaaccatgagccgccagtttagttctcagtctgcatttagctcgaggagcaggagggcctatagctccaggtcttcatcaggctttggaggtgggagacaggctctggtgtctgtgag

SEQ ID NO: 23 - DNA, 31 bp, Artificial Sequence
  primer
attaggtaccgcagccgaaggattttagtgc

SEQ ID NO: 24 - DNA, 32 bp, Artificial Sequence
  primer
agtaggatccggagcaaggtagagtaagggaa

SEQ ID NO: 25 - DNA, 32 bp, Artificial Sequence
  primer
agtcggtaccttctattgctggtgtctgtctc

SEQ ID NO: 26 - DNA, 30 bp, Artificial Sequence
  primer
tagtgacgtcctgactggctttagcccctt

SEQ ID NO: 27 - DNA, 37 bp, Artificial Sequence
  primer
aaagacgtcgccttagagagaggtgagagcaaagaca

SEQ ID NO: 28 - DNA, 36 bp, Artificial Sequence
  primer
ggactgacgtcaagatttattagtgcgttttggtgc

SEQ ID NO: 29 - DNA, 30 bp, Artificial Sequence
  primer
caacccgggcagaagcactggtagcaagga

SEQ ID NO: 30 - DNA, 30 bp, Artificial Sequence
  primer
attcgacgtctcgcctcagggatccgacct

SEQ ID NO: 31 - DNA, 35 bp, Artificial Sequence
  primer
tctagagtcgacctgcaggcatgcaagcttcttgt

SEQ ID NO: 32 - DNA, 18 bp, Artificial Sequence
  primer
gggtcgacgcccatggaa

SEQ ID NO: 33 - DNA, 21 bp, Artificial Sequence
  primer
ctggtcgacacccgggaattc

SEQ ID NO: 34 - DNA, 91 bp, Artificial Sequence
  primer
ccgggatccaacaacaacaacaaccctgcggtccaccacggccgatatcacggccgtggtggaccgcagggcaacaacaacaacaacggat

SEQ ID NO: 35 - DNA, 91 bp, Artificial Sequence
  primer
ccgttgttgttgttgttgccctgcggtccaccacggccgtgatatcggccgtggtggaccgcagggttgttgttgttgttggatcccggat

SEQ ID NO: 36 - DNA, 2039 bp, Homo sapiens
  misc_feature (3)..(3): n is a, c, g, or t
cgntgctcgttacttagcttgttaccatggatctgcccgtgggccccggcgcggcggggcccagcaacgtcccggccttcctgaccaagctgtggaccctcgtgagcgacccggacaccgacgcgctcatctgctggagcccgagcgggaacagcttccacgtgttcgaccagggccagtttgccaaggaggtgctgcccaagtacttcaagcacaacaacatggccagcttcgtgcggcagctcaacatgtatggcttccggaaagtggtccacatcgagcagggcggcctggtcaagccagagagagacgacacggagttccagcacccatgcttcctgcgtggccaggagcagctccttgagaacatcaagaggaaagtgaccagtgtgtccaccctgaagagtgaagacataaagatccgccaggacagcgtcaccaagctgctgacggacgtgcagctgatgaaggggaagcaggagtgcatggactccaagctcctggccatgaagcatgagaatgaggctctgtggcgggaggtggccagccttcggcagaagcatgcccagaacagaaagtcgtcaacaagctcattcagttcctgatctcactggtgcagtcaaaccggatcctgggggtgaagagaaagatccccctgatgctgaacgacagtggctcagcacattccatgcccaagtatagccggcagttctccctggagcacgtccacggctcgggcccctactcggccccctccccagcctacagcagctccagcctctacgcccctgatgctgtggccagctctggacccatcatctccgacatcaccgagctggctcctgccagccccatggcctcccccggcgggagcatagacgagaggcccctatccagcagccccctggtgcgtgtcaaggaggagccccccagcccgcctcagagcccccgggtagaggaggcgagtcccgggcgcccatcttccgtggacaccctcttgtccccgaccgccctcattgactccatcctgcgggagagtgaacctgcccccgcctccgtcacagccctcacggacgccaggggccacacggacaccgagggccggcctccctcccccccgcccacctccacccctgaaaagtgcctcagcgtagcctgcctggacaagaatgagctcagtgaccacttggatgctatggactccaacctggataacctgcagaccatgctgagcagccacggcttcagcgtggacaccagtgccctgctggacctgttcagcccctcggtgaccgtgcccgacatgagcctgcctgaccttgacagcagcctggccagtatccaagagctcctgtctccccaggagctccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctagaggccccggaggagctgggccagccgcccacccccacccccagtgcagggctggttttggggaggcagggcagcctcgcggttttgggcactggtgggtcggccgccatagccccagtaggacaaacgggctcgggtctgggcagcacctctggtcaggagggtcaccctggcctgccagtctgccttcccccaaccccgtgtcctgtggtttggttggggcttcacagccacacctggactgaccctgcaggttgttcatagtcagaattgtattttggatttttacacaactgtcccgttccccgctccacagagatacacagatatatacacacagtggatggacggacaagacaggcagagatctataaacagacaggctttaaaaaaaaaaaaaaaaaaaaaaaaaaaaaaaaaaaccc

SEQ ID NO: 37 - DNA, 29 bp, Artificial Sequence
  primer
gacggtaccccgaccttgacagcagcctg

(19) United States
(12) Patent Application Publication
(10) Pub. No.: US 2024/0229065 A1
(43) Pub. Date: Jul. 11, 2024

(54) REPLICATION-COMPETENT CONTROLLED HERPESVIRUSES EXPRESSING A SARS COV-2 ANTIGEN

(71) Applicant: HSF Pharmaceuticals SA, La Tour-de-Peilz (CH)

(72) Inventor: Richard W Voellmy, La Tour-d-Peilz (CH)

(73) Assignee: HSF Pharmaceuticals SA, La Tour-d-Peilz (CH)

(21) Appl. No.: 17/803,699

(22) PCT Filed: Apr. 23, 2021
(86) PCT No.: PCT/EP2021/060640

§ 371 Date: Oct. 19, 2022

Related U.S. Application Data
(60) Provisional application No. 63/101,296, filed on Apr. 24, 2020.

(51) Int. Cl. C12N 15/86; A61K 39/00; A61K 39/215; A61P 31/14; C07K 14/005

(52) U.S. Cl. CPC C12N 15/86; A61K 39/215; A61P 31/14; C07K 14/005; A61K 2039/5254; A61K 2039/5256

(57) ABSTRACT

The present disclosure relates to replication-competent controlled herpesviruses whose transient replication in an inoculation site of a subject can be activated by the delivery of an appropriate heat dose to the inoculation site region. In related recombinant viruses, activation requires delivery of a heat dose in the presence in the inoculation site of an effective concentration of a small-molecule regulator. The viruses are engineered to express an antigen from a SARS CoV-2 virus and are expected to induce strong and balanced immune responses against the SARS CoV-2 antigen in subjects to which they are administrated.

### CROSS-REFERENCE TO RELATED APPLICATIONS

This application is the U.S. national stage application of International Patent Application No. PCT/EP2021/060640, filed Apr. 23, 2021, which claims the benefit of U.S. Provisional Application No. 63/101,296, filed 24 Apr., 2020, which is incorporated herein by reference in its entirety; including any drawings.

### REFERENCE TO THE SEQUENCE LISTING

The present application is being filed along with a Sequence Listing in electronic format. The Sequence Listing is provided as a file entitled “Seq-List-replace-2.txt”, created Aug. 9, 2023, which is 13,239 bytes in size. The information in the electronic format of the Sequence Listing is incorporated herein by reference in its entirety.

### INTRODUCTION

COVID-19 is a respiratory disease that has recently emerged. The causative SARS CoV-2 viruses appear to be closely related to the SARS CoV-1 viruses that were responsible for a disease outbreak in 2003. Vaccines against the disease caused by SARS CoV-1 were developed but, because the disease could be contained, were not tested in the human population. Hence, their efficacy remains unknown. Several vaccines that are highly efficacious against currently dominant SARS CoV-2 variants were developed and are being administered to populations worldwide. Coronaviruses are RNA viruses and as such are subject to antigenic drift (and, possibly, antigenic shift caused by recombinational events). In this respect, they resemble influenza viruses for which no durable vaccine has been developed to this date. Current influenza vaccines are only moderately efficacious and need to be updated annually to assure some degree of protection. Analogously, there is reason to believe that the newly developed SARS CoV-2 vaccines will lose efficacy as the virus drifts. It is anticipated that additional vaccines against COVID-19 need to be developed that elicit a potent and balanced immune response in an immunized subject as well as are tolerant to the maximal extent possible to antigenic drift. Vaccines based on replication-competent controlled herpesviruses (RCCHV) may be capable of serving this need. The present disclosure relates to RCCHVs expressing antigens of SARS CoV-2 and their uses in vaccines or for vaccination against COVID-19 and, possibly, other coronavirus-induced disease. As used herein, the term RCCHV relates to recombinant herpesviruses whose efficient but spatially and temporally limited replication can be activated deliberately and that are essentially nonreplicating in the absence of activation. Certain replication-competent controlled viruses of this type were disclosed, e.g., in U.S. Pat. Nos. 7,906,312, 8,137,947, and U.S. patent application no. 10,478,486.

### SUMMARY OF THE INVENTION

The present disclosure relates to a replication-competent controlled herpesvirus (RCCHV) in which a first replication-essential viral gene is controlled, directly or indirectly, by a first exogenous promoter that is a nucleic acid sequence which acts as a heat shock promoter. Furthermore, the RCCHV carries in its genome an expressible gene for an antigen of a SARS CoV-2 virus, i.e., it comprises a second exogenous promoter and a functionally linked exogenous gene for an antigen of a SARS CoV-2 virus. The term “exogenous” is meant to indicate that the genetic element it relates to is introduced into an alpha-herpesvirus that serves as the backbone for the construction of the RCCHV. The first exogenous promoter is a heterologous promoter, i.e., a promoter that is not present as a whole in the backbone herpesvirus. The first exogenous promoter can be a naturally occurring heat shock promoter or a synthetic heat shock promoter created by the insertion of so-called heat shock elements into an otherwise not-heat-activated promoter. The latter heat-activated promoters are acted upon primarily by heat shock transcription factor 1 (HSF1), a transcription factor that is ubiquitously present in mammalian cells and that is activated when the cells are exposed to sublethal heat or another proteotoxic stress. The first exogenous promoter can directly control the expression of the first replication-essential gene provided that it is functionally linked to the latter gene. To achieve a more persistent (controlled) expression of the first replication-essential gene, the first exogenous promoter can be employed to control the first replication-essential gene indirectly. In this case, the first exogenous promoter is functionally linked to a (exogenous) gene for a heterologous transactivator, and this promoter-gene cassette is inserted in the genome of the backbone alpha-herpesvirus. In the resulting recombinant, the native promoter of the first replication-essential gene is replaced by an exogenous transactivator-responsive promoter. Hence, the first replication-essential viral gene is functionally linked to a transactivator-responsive promoter. To obtain a more sustained expression of the heterologous transactivator, its expression can be subjected to the control of a nucleic acid sequence that not only acts as a heat shock promoter but also as a transactivator-responsive promoter. This nucleic acid sequence may comprise a promoter that contains recognition sequences for both HSF1 and the transactivator. Alternatively, it can comprise a promoter cassette containing two separate promoters, a heat shock promoter and a transactivator-responsive promoter.

The antigen of a SARS CoV-2 virus can be a viral membrane protein (M), an envelope protein (E), a spike protein (S), a spike-like surface protein called hemagglutinin esterase (HE), a nucleocapsid protein (N) or a non-structural protein (nsp1-16), or parts of the latter proteins. Preferably, it is a spike protein. The antigen-encoding gene is driven by a second exogenous promoter. Any suitable promoter may be employed, including a promoter isolated from the backbone alpha-herpesvirus or another herpesvirus. A second copy of the first exogenous promoter may also be used. A preferred promoter is the cytomegalovirus (CMV) immediate early promoter. In RCCHVs comprising a heterologous transactivator, the promoter controlling the SARS CoV-2 antigen gene can be a transactivator-responsive promoter.

Subsequent to its administration to a site of the body of a mammalian subject (referred to as the inoculation site or the administration site), the RCCHV is induced to replicate by a localized administration of an appropriate heat dose to the inoculation site. Heat can be focused. Hence, the RCCHV can be activated to replicate only in the inoculation site region, minimizing any pathological effects that may be associated with disseminated replication of the virus. Subsequent to its heat activation, the RCCHV will replicate with a high efficiency in the heated region. This replication is transient and will end with the lysis of the infected cells in the heated region. Hence, virus replication is also limited in time/extent, further decreasing the possibility of untoward pathological effects. Over the ensuing weeks, the virus is cleared from the body of the mammalian subject, except from sensory and, depending on the virus administration site, cranial nerve ganglia where it survives in a latent form. Although heat shock promoters suitable for use in the RCCHVs are only highly active at temperatures exceeding those that are reached during pyrexia, it cannot be excluded that low-level systemic replication of an RCCHV may occur in a subject that experiences a high fever, is intoxicated or engages in high-intensity exercise shorty after virus inoculation. Similarly, the possibility of a low rate of reactivation from latency cannot be excluded, although this possibility should be further mitigated by the immunity induced in the subject by the RCCHV.

To further reduce the possibility of inadvertent replication of the RCCHV, one or more additional safety mechanisms can be introduced into the RCCHV.

One such mechanism that is aimed primarily at precluding reactivation of latently present RCCHV employs a tissue-specific promoter (also referred to a tissue-selective promoter). Hence, in this embodiment, the RCCHV further comprises a third exogenous promoter that replaces the resident promoter of a second replication-essential viral gene. Hence, the second replication-essential viral gene is placed under the control of (is functionally linked to) the third exogenous promoter. The third exogenous promoter is a promoter that is active in cells in the selected inoculation site of a mammalian subject (i.e., the site to which the RCCHV is to be administered) but is essentially inactive in the cells of the nerve ganglia (or, more generally, of nervous tissue) of the mammalian subject. The terms “active” and “essentially inactive” refer to transcript levels of the gene that is naturally controlled by the chosen third exogenous promoter as published in professional databases, of which BioGPS is a preferred database (biogps.org). A suitable third exogenous promoter is a promoter of a mammalian gene whose level of expression (transcript level) in the tissue that is selected for administration of the RCCHV (in which tissue the inoculation site is located) is at least about 50 times, more preferably at least about 100 times and most preferably at least about 500 times higher than in (cells of) nerve ganglia and, typically, other nerve tissue. In addition, the level of expression (transcript level) of the latter mammalian gene in nerve ganglia and, typically, in other nervous tissue is less than about three times the median level of expression (transcript level) of all tissues examined. Preferably, it is close to or less than the median transcript level of all tissues examined. It is noted that a transcript level corresponding to the median transcript level of a gene that is expressed in a highly tissue-selective fashion corresponds to a level that is close to or at the limit of detection, and a transcript level of three times the median transcript level is still extremely low. Hence, the promoter of such a gene is “essentially inactive”. The term “essentially inactive” may be replaced by the term “inactive”, whereby the meaning of the latter term would be defined by the above limitations on transcript levels.

The primary targets of the alpha-herpesviruses that can serve as backbones for the construction of RCCHVs of the present disclosure are epithelial cells. Hence, a selected inoculation site for an RCCHV of the present disclosure can be a cutaneous site or a mucosal membrane of a mammalian subject. A preferred inoculation site can be an epidermal site, preferably an epidermal site in an extremity of a mammalian subject. With these preferences, databases can be mined for genes that are active in the epidermis (or the skin or a mucosal membrane) but are essentially inactive in nerve ganglia, e.g., the dorsal root ganglia, or are essentially inactive in nervous tissue generally. Such a search uncovers, e.g., the keratin-1 gene as is discussed further below. The promoter of this gene can serve as a third heterologous promoter for an RCCHV that is intended for administration to a skin site of a mammalian subject.

A further mechanism is aimed at precluding both inadvertent systemic replication of an RCCHV shortly after administration to a mammalian subject as well as reactivation of latently present RCCV. In embodiments employing this mechanism, the heterologous transactivator that is expressed by the respective RCCHV is a regulated transactivator that is enabled to mediate transcription from a transactivator-responsive promoter by an activating interaction with an appropriate small-molecule regulator (SMR). An RCCHV of the present disclosure that carries a gene for an SMR-activated transactivator is referred to as a heat- and SMR-controlled RCCHV, and an RCCHV that does not express a regulated transactivator is referred to as a heat-controlled RCCHV. In principle, the regulated transactivator can be any transactivator that can be activated by an SMR. Preferred is a transactivator for which an SMR is available that has no undue toxicity in the mammalian subject to which RCCHV is to be administered. As discussed further below, there are several transactivator/SMR combinations that may satisfy the latter requirement. Preferred is a regulated transactivator that contains a truncated ligand-binding domain from a progesterone receptor and is activated by a narrow class of progesterone receptor antagonists (antiprogestins). Typical for the class of antiprogestins that activate the latter transactivator are mifepristone and ulipristal. The most preferred transactivator of this type is GLP65. Burcin et al. (1999) (Proc Natl Acad Sci USA (1999) 96: 355-60); Ye et al. (2002) (Meth Enzymol (2002) 346: 551-61).

Yet another mechanism aimed at precluding both systemic replication of the RCCHV shortly after administration to a mammalian subject as well as reactivation of latently present RCCV employs a repressor of the first replication-essential gene. Hence, in an embodiment employing this mechanism, the RCCHV further comprises, inserted in its genome, an expressible gene for a repressor of the first replication-essential gene. For example, in an RCCHV in which the first replication-essential viral gene is directly controlled by a nucleic acid sequence that acts as a heat shock promoter, the repressor can be a modified (constitutively trimeric) HSF1 in which the activation domain has been replaced by a repressor domain such as a KRAB domain. In an RCCHV in which the expression of the first replication-essential gene is controlled by a transactivator-responsive promoter, the repressor can by a chimeric protein comprising the DNA-binding domain of the heterologous transactivator and a repressor domain such as a KRAB domain. The promoter that drives the expression of the latter repressors will be a promoter that is not active (or essentially inactive) in the tissue of the intended inoculation site but is active in nervous tissues and, possibly, other tissues. Alternatively, in an RCCHV that employs a heterologous transactivator for controlling the expression of the first replication-essential gene, the repressor could be a chimeric protein comprising the DNA-binding of the heterologous transactivator, an appropriate SMR-binding domain and a repressor domain such as a KRAB domain. The promoter that drives the expression of the latter repressor could be a ubiquitously active promoter such as, e.g., a CMV early promoter or a mammalian ROSA promoter. Repression would be triggered by the addition/administration of an effective amount of the SMR.

An RCCHV of the present disclosure can be derived from a herpes simplex virus 1 (HSV-1), a herpes simplex virus 2 (HSV-2), or a varicella zoster virus (VZV). The latter herpesvirus will typically be a natural isolate.

In an RCCHV of the present disclosure that has been derived from an HSV-1 or HSV-2, the viral gene ICP47 can be deleted or rendered nonfunctional. The product of this gene binds to transporters associated with antigen processing (TAP), interfering with the presentation of antigens to MHC class I molecules and, consequently, with immune recognition by cytotoxic T-lymphocytes.

Also encompassed by the present disclosure is a vaccine composition to be used for prophylactic or therapeutic vaccination of a mammalian subject against COVID-19 or other coronavirus-induced disease, which composition comprises an effective amount of an RCCHV of the present disclosure. In the case of a heat- and SMR-controlled RCCHV, a composition comprising an effective amount of an SMR that is capable of activating the heterologous transactivator can be co-administered with the vaccine composition or can be administered separately, including by a different route. For example, a vaccine composition can be administered topically to a skin site of a mammalian subject and an SMR-comprising composition can be co-administered to the same site or can be administered systemically (e.g., per os). Alternatively, a vaccine composition comprising an effective amount of a heat- and SMR-controlled RCCHV can further comprise an effective amount of an SMR that is capable of activating the heterologous transactivator comprised in the RCCHV. If it is administered as an aqueous solution or suspension, the vaccine composition will typically comprise a pharmaceutically acceptable carrier or excipient.

Also encompassed by the present disclosure are uses of RCCHVs of the present disclosure (or of compositions comprising an effective amount of an RCCHV of the present disclosure and a pharmaceutically acceptable carrier or excipient) for therapeutic or prophylactic vaccination of mammalian subjects against COVID-19 or other coronavirus-induced disease. Also provided herein is a replication-competent controlled herpesvirus (e.g. for delivering an antigen of a SARS CoV-2 virus) comprising inserted in the genome of an alpha-herpesvirus: (a) a first exogenous promoter that is a nucleic acid sequence that acts as a heat shock promoter, the first promoter controlling the expression of a first replication-essential gene of the alpha-herpesvirus, and (b) a second exogenous promoter and a functionally linked exogenous gene for an antigen of a SARS CoV-2 virus. Yet further provided herein is a replication-competent controlled herpesvirus capable of delivering an antigen of a SARS CoV-2 virus, comprising inserted in the genome of an alpha-herpesvirus:(a) a first exogenous promoter that is a nucleic acid sequence that acts as a heat shock promoter, the first promoter controlling the expression of a first replication-essential gene of the alpha-herpesvirus, and (b) a second exogenous promoter and a functionally linked exogenous gene for an antigen of a SARS CoV-2 virus.

### BRIEF DESCRIPTION OF FIGURES

FIG. 1 presents a nucleotide sequence for the promoter and RNA leader region of the mouse keratin 1 (KRT1) gene (retrieved from the Eukaryotic Promoter Database EPD (epd.epfl.ch)). The sequence includes 1200 nucleotide of 5′ untranscribed sequence and the first 200 nucleotides of the transcript. The start of transcription site is bolded.

FIG. 2 presents a nucleotide sequence for the promoter and RNA leader region of the mouse keratin 77 (KRT77) gene (retrieved from the Eukaryotic Promoter Database EPD (epd.epfl.ch)). The sequence includes 1200 nucleotide of 5′ untranscribed sequence and the first 200 nucleotides of the transcript. The start of transcription site is bolded.

FIG. 3 shows the nucleotide sequence of human HSF1 present in plasmid CMV-hHSF1. The protein-coding sequence is underlined.

### DETAILED DESCRIPTION

Unless otherwise defined below or elsewhere in the present specification, all terms shall have their ordinary meaning in the relevant art.

“Replication of virus” or “virus/viral replication” are understood to mean multiplication of viral particles. Replication is often measured by determination of numbers of infectious virus, e.g., plaque-forming units of virus (pfu). However, replication can also be assessed by biochemical methods such as methods that determine amounts of viral DNA, e.g., by a real-time PCR procedure, levels of viral gene expression, e.g., by RT-PCR of gene transcripts, etc. However, it is understood that marginal increases in levels of viral DNA or viral gene transcripts or protein products may not translate in corresponding marginal increases in virus replication due to threshold effects.

A “small-molecule regulator” or “SMR” is understood to be a low molecular weight ligand of a transactivator used in connection with this disclosure. The SMR is capable of activating the transactivator. The SMR is typically, but not necessarily, smaller than about 1000 Dalton (1 kDa).

The term “transactivator” or “heterologous transactivator” is used herein to refer to a non-viral and, typically, engineered transcription factor that can positively affect transcription of a gene controlled by a transactivator-responsive promoter.

A “small-molecule regulator (SMR)-activated transactivator” or “heterologous small-molecule regulator (SMR)-activated transactivator” is a non-viral and, typically, engineered transcription factor that when activated by the appropriate small-molecule regulator (SMR) positively affects transcription of a gene controlled by a transactivator-responsive promoter.

A “transactivator-responsive promoter” is a promoter that contains one or more sequence elements that can be bound by a transactivator and that is essentially inactive prior to being bound by the transactivator.

“Activated” when used in connection with a transactivated gene generally means that the rate of expression of the gene is measurably greater after activation than before activation or, when used in connection with a controllable heterologous promoter means that the transcription-enhancing activity of the promoter is measurably greater after activation than before activation.

This generic definition is not meant to contradict the more specific limitations that apply to the activity of genes that are controlled by a tissue-specific promoter as defined above. When used in connection with a SMR-activated transactivator, “active” or “activated” refers to a transactivation-competent form of the transactivator. The transactivator is rendered transactivation-competent by the binding of the appropriate SMR. When used in connection with an RCCHV, “activated” refers to an RCCHV that has been triggered to undergo at least one round of replication.

Herein, a virus, whose genome includes a foreign (heterologous) non-viral or viral gene or has been modified otherwise, is referred to as a “virus”, a “viral vector” or a “recombinant”.

A “replication-competent controlled virus” is a recombinant virus whose replicative ability is under deliberate control (i.e., whose replication can be triggered by a deliberate intervention). In such a virus, replication of at least one replication-essential viral gene is under the control of a gene switch that can be deliberately activated. A “replication-competent controlled herpesvirus”, abbreviated as “RCCHV”, is a replication-competent controlled virus that typically has been derived from a wildtype (natural isolate) alpha-herpesvirus.

A “heterologous promoter” is a promoter that, as a whole, does not naturally occur in the herpesvirus that serves as the backbone for the construction of a replication-competent controlled virus. The possibility that such a heterologous promoter also contains elements of a viral promoter is encompassed by the term.

The term “exogenous” when associated with a promoter or gene is meant to indicate that the genetic element it relates to is introduced into a herpesvirus that serves as the backbone for the construction of an RCCHV.

A “replication-essential gene” or a “gene required for efficient replication” is arbitrarily defined herein as a viral gene whose loss of function diminishes replication efficiency by a factor of ten or greater. Replication efficiency can be estimated, e.g., in a (single-step) growth experiment. For many viruses it is well known which genes are replication-essential genes. For herpesviruses see, e.g., Nishigawa (1996) (Nagoya L Med Sci (1996) 59: 107-19).

The terms “inoculation site” and “administration site” are used interchangeably. By their ordinary meaning, the terms refer to the point of administration (or points of administration in case of a needle patch) of an RCCHV or a composition comprising an RCCHV. Operationally, the terms also encompass an area surrounding the point(s) of administration as virus will diffuse from the point of administration or will be forced to distribute in the case of an injection of a liquid containing an RCCHV. In the case of administration to a skin site, this area may be defined arbitrarily as a circular (nonplanar) area with a radius of about 10 cm or, more narrowly, 5 cm centered around the point(s) of administration. In the case of administration to a mucosal membrane, the area can encompass the lining of the entire cavity harboring the target mucosal membrane, e.g., the nose, the mouth, the vagina or the lungs. To reflect these facts, the terms “inoculation site region” or “administration site region” are also employed.

The terms “selected inoculation site” or “selected administration site” refer to the inoculation site or administration site that a designer (and, consequently, a producer or distributor) of an RCCHV vaccine decided to target, which decision drove the construction of an RCCHV that is capable of replicating efficiently in that site. Some of the RCCVs exemplified herein were designed with the intention of administering them to a site in the skin or a mucosal membrane of a subject. Therefore, tissue-selective exogenous promoters that exhibit elevated activities in skin and mucosal tissues were employed in these RCCHVs.

An “effective amount of a replication-competent controlled herpesvirus or (RCCHV)” is an amount of virus that upon single or repeated administration to a subject followed by activation confers a therapeutic effect on the treated subject, at a reasonable benefit/risk ratio applicable to any medical treatment. When used in the context of prophylaxis or prevention, an “effective amount” of an RCCHV of the present disclosure is meant to be an amount which, when administered (and thereafter activated) once or multiple times over the course of a prophylactic (e.g., vaccination) regime, confers a desired prophylactic effect on the treated subject. In general, what is an “effective amount” will vary depending on the route of administration as well as the possibility of co-usage with other agents. It will be understood that the total or fractional dosage of compositions of the present disclosure comprising an RCCHV will be decided by a competent regulatory authority or by an attending physician within the scope of sound medical judgment. The specific effective dose level may be based on a variety of factors including the activity of the RCCHV employed; the specific composition employed; the age, body weight, general health, sex and diet of the patient; the time of administration, route of administration, and rate of elimination of the specific RCCHV employed; the duration of the treatment; drugs used in combination or contemporaneously with the RCCHV employed; and like factors well known in the medical arts. The latter factors will be considered in the context of therapeutic applications of replication-competent controlled viruses as well as in the context of prophylactic or preventative applications.

An “effective amount of a small-molecule regulator (SMR)” is an amount that when administered to a subject by a desired route is capable of co-activating a heat- and SMR-controlled RCCHV of the present disclosure with which the subject concurrently is, has been or will be inoculated to undergo at least one round of replication in the inoculation site.

A “subject” or a “mammalian subject” is a mammalian animal or a human person.

“Promoter of a heat shock gene”, “heat shock gene promoter” and “heat shock promoter” are used synonymously. A “nucleic acid that acts as a heat shock promoter” can be a naturally found heat shock promoter or a synthetic promoter created by the addition to a promoter other than a heat shock promoter of sequence elements of the type present in heat shock promoters, which elements confer heat activation on a functionally linked gene.

A “heat shock gene” is defined herein as any gene, from any eukaryotic organism, whose activity is enhanced when the cell containing the gene is exposed to a temperature above its normal growth temperature. Typically, such genes are activated when the temperature to which the cell is normally exposed is raised by 3-10° C. Heat shock genes comprise genes for the “classical” heat shock proteins, i.e., HSP110, HSP90, HSP70, HSP60, HSP40, and HSP20-30. They also include other heat-inducible genes such as genes for MDR1, ubiquitin, FKBP52, heme oxidase and other proteins. The promoters of these genes, the “heat shock promoters”, contain characteristic sequence elements referred to as heat shock elements (HSE) that consist of perfect or imperfect sequence modules of the type NGAAN or AGAAN (“N” standing for any nucleotide), which modules are arranged in alternating orientations (Amin et al. (1988) (Mol Cell Biol (1988) 8: 3761-3769); Xiao and Lis (1988) (Science (1988) 239: 1139-1142); Fernandes et al. (1994) (Nucleic Acids Res (1994) 22: 167-173). These elements are highly conserved in all eukaryotic cells such that, e.g., a heat shock promoter from a fruit fly can be functional and heat-regulated in a frog cell (Voellmy and Rungger (1982) (Proc Natl Acad Sci USA (1982) 79: 1776-1780). HSE sequences are binding sites for heat shock transcription factors (HSFs; reviewed in Wu (1995) (Annu Rev Cell Dev Biol (1995) 11: 441-469). The factor primarily responsible for activation of heat shock genes in vertebrate cells exposed to heat or a proteotoxic stress is heat shock transcription factor 1 (abbreviated as “HSF1”) (Baler et al. (1993) (Mol Cell Biol (1993) 13: 2486-2496); McMillan et al. (1998) (J Biol Chem (1998) 273: 7523-7528). Preferred promoters for use in RCCHVs discussed herein are those of inducible HSP70 genes. A particularly preferred heat shock promoter is the promoter of the human HSP70B gene (Voellmy et al. (1985) (Proc Natl Acad Sci USA (1985) 82: 4949-4953).

“Vaccine” typically refers to compositions comprising microbes that are killed, replication-defective or otherwise attenuated, or to components of such microbes. Herein, the term is expanded to also include compositions comprising RCCHVs that when activated can induce an immune response in a subject to which they are administered.

Current thought appears to be that in order to be effective or more effective, respectfully, improved vaccine candidates for preventing or treating diseases such as herpes, HIV, tuberculosis, COVID or influenza need to elicit a balanced immune response that also includes a powerful effector T cell response. The present disclosure relates to RCCHVs that, upon activation, replicate with efficiencies that approach those of the respective wildtype viruses. It is hypothesized that these recombinant viruses and any heterologous protein they express will be potent immunogens that elicit balanced, broad immune responses.

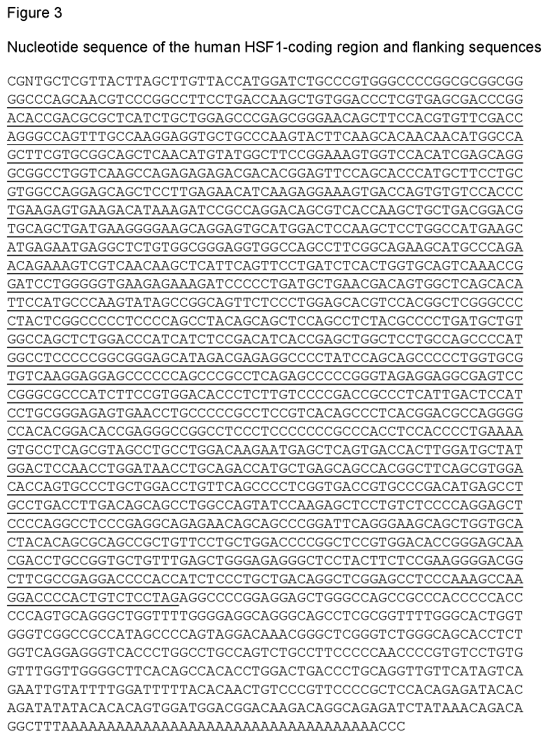

To obtain an RCCHV of the present disclosure, a wild type HSV-1, HSV-2 or varicella zoster virus (VZV) can be genetically altered by placing at least one replication-essential viral gene under the control of a gene switch that has a broad dynamic range, i.e., that essentially functions as an on/off switch. The gene switch can be a highly heat-inducible heat shock promoter or a nucleic acid sequence that functions as such a heat shock promoter (the promoter also being referred to herein as “first exogenous promoter”). Alternatively, it can be a highly heat-inducible heat shock promoter (or nucleic acid sequence that functions as such a heat shock promoter) that drives the expression of a gene for a heterologous transactivator, and the heterologous transactivator controls the expression of a replication-essential gene that is functionally linked to a transactivator-responsive promoter. As heat shock promoters can only be transiently activated, it may be advantageous to include an element that permits autoactivated synthesis of the transactivator. Hence, the transactivator gene may be controlled by a nucleic acid sequence that acts both as a heat shock promoter and as a transactivator-responsive promoter. Such a nucleic acid sequence is present in several of the exemplified RCCHVs and is specifically discussed in the Example that relates to the construction of HSV-GS1/3. See also FIG. 3 in Vilaboa et al. (2005) (Mol Ther (2005) 12: 290-298).

Any transactivator may be employed as long as it is transcriptionally competent when synthesized in a mammalian cell (i.e., is constitutively active) and comprises a DNA-binding domain that specifically binds to a DNA sequence element that is present in a transactivator-responsive promoter. Advantageously, the transactivator is an SMR-activated transactivator that is active in the presence of the SMR but is essentially inactive in its absence. To construct an RCCHV that relies on a highly heat-inducible heat shock promoter (or nucleic acid sequence that functions as such a heat shock promoter) as the gene switch, the resident promoter of at least one replication-essential gene in a herpesvirus is replaced with the latter heat shock promoter. To construct an RCCHV in which one or more replication-essential genes are controlled by a transactivator that is expressed from a heat shock promoter-driven gene, a herpesvirus is modified to contain the expressible gene for the transactivator in a location in the viral genome in which an insertion of such a gene does not interfere with virus function, in particular with replication efficiency. The UL43/44 and UL37/38 intergenic regions of HSV-1 are such locations. The promoters of the one or more replication-essential genes are replaced by transactivator-responsive promoters. The resulting recombinant viruses are referred to as heat-controlled or, when co-activated by an SMR, as heat- and SMR-controlled RCCHVs.

The RCCHVs of the present disclosure further contain an inserted gene for an antigen of a SARS CoV-2 virus. In the examples shown below, an antigen-expressing gene was inserted into the UL37/38 intergenic region by recombination. The antigen of a SARS CoV-2 virus can be a viral membrane protein (M), an envelope protein (E), a spike protein (S), a spike-like surface protein called hemagglutinin esterase (HE), a nucleocapsid protein (N) or a non-structural protein (nsp1-16), or parts (segments) of the latter proteins. Preferably, it is a spike protein or segments thereof. Most preferably, it is a complete spike protein. Nucleotide sequences of complete SARS CoV-2 genomes can be obtained from the U.S. National Library of Medicine, National Center for Biotechnology Information. The antigen-encoding gene is driven by a heterologous or a herpesvirus promoter (also referred to as “second exogenous promoter”). Any suitable promoter may be employed. A preferred promoter is the cytomegalovirus (CMV) immediate early promoter. In RCCHVs comprising a heterologous transactivator, the promoter controlling the SARS CoV-2 antigen gene can be a transactivator-responsive promoter.

HSV-1, HSV-2 or VZV are known to preferentially target epithelial cells and to establish latency in cells of nerve ganglia, e.g., in cells of the dorsal root ganglia. In certain embodiments, RCCHVs of the present disclosure are engineered to replicate efficiently in epithelial cells (and derived cells), e.g., epidermal cells of the skin, but to not replicate or replicate only minimally in cells of nerve ganglia (and other tissues to the extent that this is achievable). This is accomplished by the use of an appropriate tissue-specific or tissue-selective (heterologous) promoter for controlling the expression of a further replication-essential gene. Hence, the construction of such an RCCHV involves the replacement in a heat-controlled or a heat- and SMR-controlled RCCHV (that carries a SARS CoV-2 antigen) of the resident promoter of a further replication-essential gene with an appropriate tissue-specific or tissue-selective (heterologous) promoter (also referred to as “third exogenous promoter”). It is understood that such an RCCHV can also be constructed by first replacing in a herpesvirus the promoter of a replication-essential gene with a tissue-specific or selective promoter and subsequently placing at least one other replication-essential gene under heat or heat and SMR control. The expressible gene for a SARS CoV-2 antigen can be inserted at any stage.

A tissue-specific or tissue-selective promoter that is appropriate for the intended use in an RCCHV of the present disclosure can be identified by mining available databases and other scientific literature (see, e.g., the BioGPS database or the article of Su et al. (2002) (Proc Natl Acad Sci USA (2002) 99: 4465-4470).

For an RCCHV of the present disclosure that is to be used as a vaccine and is to be administered to a site in the skin of a human subject, a promoter that is highly active in any or all layers of the human skin but not (or only minimally) in cells of nerve ganglia may be selected for driving the expression of one or more replication-essential viral genes. A particular promoter that may be employed is the human keratin 1 (KRT1) promoter. Edqvist et al. (2015) (J Histochem Cytochem (2015) 63: 129-41). Inspection of the BioGPS database (biogps.org) reveals that this promoter is essentially only expressed in skin (and other epithelia as discussed below). No expression is evident in nerve ganglia and other nerve cells (Table 1). The mouse KRT1 promoter can also be used as it is inactive in human (and murine) nerve cells. Another useful promoter is that of the human KRT10 gene. Transcript levels are far higher in the skin than in any other tissue/organ. Essentially no expression occurs in nerve ganglia and other nerve cells. For an RCCHV vaccine to be administered to the skin of a murine subject, a suitable promoter can be that of the mouse KRT77 gene. The latter gene is highly active in the mouse epidermis and weakly active in the umbilical cord. Essentially no expression occurs in the dorsal root ganglia, other nerve cells and other adult tissues. Another suitable promoter is the mouse KRT10 promoter for which essentially no activity has been demonstrated in nerve cells. That it is also active in the stomach and the umbilical cord should not significantly detract from its usefulness in vaccine applications. The mouse filaggrin gene (FLG) has a similar expression profile. The human filaggrin promoter appears to be more selectively active in the skin. The nucleotide sequences of the latter promoters can be found in the Eukaryotic Promoter Database (epd.epfl.ch) and elsewhere.

For an RCCHV-based human vaccine that is co-directed against genital herpes or other sexually transmitted diseases and that is to be administered to the vaginal mucosal epithelium, reference is made to Borgdorff et al. (2016) (Mucosal Immunol (2016) 9: 621-33). The latter publication reports that the KRT1, KRT4, KRT5, KRT6A, KRT10 and KRT13 genes are abundantly expressed in the epithelial layer of the vagina. Preferred promoters for use in RCCHVs suitable for the latter applications are those of the KRT1, KRT4 and KRT13 genes that exhibit the most selective mucosal activity but appear to have essentially no activity in nerve cells. A suitable promoter to be used in an RCCHV intended for administration to the lungs is the human KRT14 promoter. This promoter is also active in the skin and in the tongue, but is essentially devoid of activity in nervous cells.

TABLE 1
Expression of KRT genes

| | | | Rel. | Rel. |
|---|---|---|---|---|
| | | | transcript | transcript |
| | | Rel. | level in | level |
| | | transcript | dorsal | over all |
| | | level | root | tissues - |
| Gene | ID | in skin | ganglia | median |
| Mouse | 16678 | 13452 | 4.7 | 4.6 |
| KRT1 | | (epidermis) | | |
| Human | 3848 | 6713 | 2.9 | 3.9 |
| KRT1 | | | | |
| Mouse | 406220 | 37271 | 4.6 | 4.6 |
| KRT77 | | | | |
| Mouse | 16661 | 92700 | 158 | 124 |
| KRT10 | | (epidermis) | | |
| Human | 3858 | 5939 | 15 | 93 |
| KRT10 | | | | |

Data from the BioGPS database

Tissue-specific or selective promoters for controlling the expression of one or more replication-essential genes in an RCCHV of the present disclosure, including the promoters specifically disclosed above, are selected because they are active in the intended target cells but essentially inactive in cells of nerve ganglia, and their use in the herpesvirus recombinants is expected to preclude or minimize the possibility of reactivation of the viruses from latency. It appears improbable that, when using such selective promoters, an unacceptable level of replication is detected in neural cells (which is a level that enables detectable reactivation from latency in a subject). However, if this occurs, it is likely that the promoter concerned is also excessively active in the target cells, i.e., highly efficient (wildtype-like) replication could be had at a considerably lower level of promoter activity. Hence, to reduce such undue viral replication in neural cells, it may be indicated to generally reduce the level of expression of the replication-essential gene(s) controlled by the promoter. Various engineering approaches to achieve this goal are known in the art. For example, protein-destabilizing elements could be introduced into the replication-essential protein, e.g., near the carboxy terminus of the protein. Well known sequence elements of this type are the so-called PEST sequences that are thought to function as proteolytic signals. Rechsteiner and Rogers (1996) (Trends Biochem Sci (1996) 21: 267-71). These sequences contain regions enriched in proline (P), glutamate (E), serine (S) and threonine (T). PEST sequences are hydrophilic stretches of at least 12 amino acids in length, with the entire region flanked by lysine (K), arginine (R) or histidine (H), but not interrupted by positively charged residues. RNA-destabilizing elements, AU-rich elements (ARE), may be added to the 3′UTR sequence of the replication-essential gene. Such elements were described in Zubiaga et al. (1995) (Mol Cell Biol (1995)15: 2219-30). See also Matoulkova et al. (2012) RNA Biol 9: 563-76. RNA- and protein-destabilizing elements have also been used in combination to dramatically reduce protein levels. Voon et al. (2005) Nucleic Acids Res 33 (3): e27. Other approaches are aimed at reducing translation efficiency. The introduction of highly stable secondary structure (hairpins) near the 5′ end of a gene can dramatically reduce translation efficiency as shown by Babendure et al. (2006) (RNA (2006) 12: 851-61). Hence, such secondary structure elements could be introduced into the replication-essential gene to reduce its expression.

The following non-limiting description illustrates how a SARS CoV-2 antigen-carrying RCCHV of the present disclosure may be employed. A composition comprising an effective amount of a heat-controlled or a heat- and SMR-controlled RCCHV of the present disclosure and, in the case of a heat- and SMR-controlled RCCHV, an effective amount of an appropriately formulated SMR is administered to a subject intradermally. Shortly after administration, a heating pad is activated and applied to the inoculation site region by either the subject or a medical practitioner. Heating at about 43.5-45.5° C. (temperature of the pad surface in contact with the skin) will be for a period of about 10-60 min. The latter heat treatment will trigger one cycle of virus replication. If another round of replication is desired, another activated pad is applied to the inoculation site region at an appropriate later time. If an immunization procedure employing a heat- and SMR-controlled RCCHV involves sequential heat treatments, SMR may also need to be administered sequentially. Alternatively, a slow-release formulation may be utilized that assures the presence of an effective concentration of the SMR in the inoculation site over the period during which viral replication is desired.

More generally, a body site to which an RCCHV of the present disclosure is administered, i.e., the inoculation site, may be heated by any suitable method. Heat may be delivered or produced in the target region by different means including direct contact with a heated surface or a heated liquid, ultrasound, infrared irradiation, or microwave or radiofrequency irradiation. As proposed in the above specific example, a practical and inexpensive solution may be offered by heating pads (or similar devices of other shapes, e.g., cylinders or cones, for heating mucosal surfaces of the nose, etc.) containing a supercooled liquid that can be triggered by mechanical disturbance to crystallize, releasing heat at the melting temperature of the chemical used. A useful chemical may be sodium thiosulfate pentahydrate that has a melting temperature of about 48° C. U.S. Pat. Nos. 3,951,127, 4,379,448, and 4,460,546. The technology is readily available and is already being used in health care products. That such heating pads are capable of activating heat shock promoters in all human skin layers has been verified experimentally. Voellmy et al. (2018) Cell Stress Chaperones 23(4): 455-466.

An “activating heat dose” is a heat dose that causes a transient activation of heat shock transcription factor 1 (HSF1) in cells at the inoculation site. Activation of HSF1 is evidenced by a detectably increased level of RNA transcripts of a heat-inducible heat shock gene over the level present in cells that did not receive the heat dose. Alternatively, it may be evidenced as a detectably increased amount of the protein product of such a heat shock gene. Moreover, an activating heat dose may be evidenced by the occurrence of replication of a heat-controlled RCCHV, in the presence of an effective concentration of an appropriate small-molecule regulator in the case of a heat- and SMR-controlled RCCHV.

An activating heat dose can be delivered to the target region generally at a temperature between about 41° C. and about 47° C. for a period of between about 1 min and about 180 min. It is noted that heat dose is a function of both temperature and time of exposure. Hence, similar heat doses can be achieved by a combination of an exposure temperature at the lower end of the temperature range and an exposure time at the upper end of the time range, or an exposure temperature at the higher end of the temperature range and an exposure time at the lower end of the time range. Preferably, heat exposure will be at a temperature between about 42° C. and about 46° C. for a duration of between about 5 min and about 150 min. Most preferably, heat treatment is administered at a temperature between about 43.5° C. and about 45.5° C. for a duration of between about 10 min and about 60 min. It is noted that it appears possible to deliver an activating heat dose within a much shorter time, i.e., within seconds or even in the sub-second range, by intense irradiation of the target region. Tolson and Roberts (2005) (Methods (2005) 35:149-157); Sajjadi et al. (2013) (Med Eng Phys (2013) 35:1406-1414).

Concerning heat- and SMR-controlled RCCHVs of the present disclosure, an SMR should satisfy a number of criteria. Most important will be that the substance is safe; adverse effects should occur at most at an extremely low rate and should be generally of a mild nature. Ideally, an SMR would belong to a chemical group that is not used in human therapy. However, before any substance not otherwise developed for human therapy could be used as an SMR in a medical application of an RCCHV, it would have to undergo extensive preclinical and clinical testing. It may be more efficacious to select a known and well-characterized drug substance that is not otherwise administered to the specific population targeted for immunization using an RCCHV. Alternatively, a known drug substance that will not need to be administered to subjects within at least the first several weeks after RCCHV-mediated treatment or immunization may be selected as an SMR. Thus, the potential low-level risk of disseminated replication of the RCCHV would be further reduced by the avoidance of administration of the drug substance during the period during which the RCCHV is systemically present. Subsequent, ideally sporadic, use of the drug substance under medical supervision will ensure that any significant inadvertent replication of an RCCHV would be rapidly diagnosed and antiviral measures could be taken without delay. In examples described herein, the SMR is a progesterone receptor (PR) antagonist or antiprogestin, e.g., mifepristone or ulipristal. Mifepristone and ulipristal fulfill the latter requirement of not typically needing to be administered shortly after virus administration. Mifepristone and ulipristal, when used in single-dose regimes, have excellent human safety records.

An effective concentration of an SMR in the inoculation site is a concentration that enables replication (at least one round) of an RCCHV in infected cells in that site. What an effective concentration is depends on the affinity of the SMR for its target transactivator. How such effective concentration is achieved and for how long it is maintained also depends on the pharmacokinetics of the particular SMR, which in turn depends on the route or site of administration of the SMR, the metabolism and route of elimination of the SMR, the subject to which the SMR is administered, i.e., the type of subject (human or other mammal), its age, condition, weight, etc. It further depends on the type of composition administered, i.e., whether the composition permits an immediate release or a slow release of the regulator. For a number of well-characterized SMR-transactivator systems, effective concentrations in certain experimental subjects have been estimated and are available from the literature. This applies to systems based on progesterone receptor, ecdysone receptors, estrogen receptors, and tetracycline repressor as well as to dimerizer systems, i.e., transactivators activated by rapamycin or analogs (including non-immunosuppressive analogs), or FK506 or analogs. For example, an effective concentration of mifepristone in rats can be reached by i.p. (intraperitoneal) administration of 5 μg mifepristone per kg body weight (5 μg/kg). Amounts would have to be approximately doubled (to about 10 μg/kg), if the SMR were administered orally. Wang, Y. et al. (1994) Proc Natl Acad Sci USA 91: 8180-84. Amounts of an SMR that, upon administration by the chosen route to the chosen site, result in an effective concentration are referred to as effective amounts of the SMR in question. How an effective amount of an SMR that results in an effective concentration can be determined is well within the skills of an artisan.

In the afore-described specific example of how the novel immunization method may be practiced, a heat- and SMR-controlled RCCHV of the present disclosure and an appropriate SMR were co-administered in a single composition. RCCHV and SMR can also be administered in separate compositions. Topical co-administration of immunizing virus and SMR appears advantageous for several reasons, including minimization of potential secondary effects of the SMR, further reduction of the already remote possibility that virus may replicate systemically during the immunization period, and minimization of the environmental impact of elimination of SMR. Notwithstanding these advantages, the SMR may be given by a systemic route, e.g., orally, which may be preferred if a formulation of the drug substance of choice is already available that has been tested for a particular route of administration. The relative timing of inoculation with RCCHV, administration of an appropriate heat dose and administration of an effective amount of SMR is derivative of the operational requirements of dual-responsive gene switch control. Typically, inoculation with immunizing virus will precede heat treatment. This is because heat activation of HSF1 is transient, and activated HSF1 returns to an inactive state within at most a few hours after activation. The dual-responsive transactivator gene present in the viral genome must be available for HSF1-mediated transcription during the latter short interval of factor activity. For the latter gene(s) to become available for transcription, the immunizing virus will have had to adsorb to a host cell, enter the cell and unravel to present its genome to the cellular transcription machinery. Although not preferred, it is possible to heat-expose the inoculation site immediately after (or even shortly before) administration of the immunizing virus. Typically, the inoculation site is heat-exposed at a time between about 30 min to about 10 h after virus administration, although heat treatment may be administered even later. Regarding administration of the SMR, there typically will be more flexibility because it will be possible to maintain an effective concentration systemically or specifically in the inoculation site region for one to several days. Consequently, SMR can be administered prior to, at the time of or subsequent to virus administration, the only requirement being that the regulator be present in an effective concentration in the inoculation site for the time needed for the target transactivator to fulfill its role in enabling viral replication. Typically, this time will correspond to that required for the completion of a round of induced virus replication. Typically, a round of virus replication will be completed within one day.

As has been alluded to before, in the novel immunization method an RCCHV may be induced to replicate once or several times. Replication may be re-induced one to several days after the previous round of replication. Such repeated replication will serve to increase viral load in the subject and to further stimulate the subject's immune system. For any round of replication to occur, the target cells that are infected with RCCHV need to receive an activating heat dose and the tissue of which the latter cells are part (the inoculation site) must contain an effective concentration of SMR (in the case of a heat- and SMR-controlled RCCHV).

Inoculation can be by any suitable route. The body site (inoculation site) to which an RCCHV is administered will typically be a cutaneous site located anywhere on the trunk or the extremities of a subject. Preferably, administration of a composition of the present disclosure comprising an RCCHV will be to a cutaneous site located on an upper extremity of the subject. Administration may also be to the lungs or airways, a mucous membrane in an orifice of a subject or a site in another tissue in which the virus is capable of replicating efficiently.

The heat- and SMR-controlled RCCHVs exemplified herein are controlled by a heat- and antiprogestin-coactivated gene switch. They express mifepristone- or ulipristal-activated chimeric transactivator GLP65 (or glp65). This transactivator comprises a DNA-binding domain from yeast transcription factor GAL4, a truncated ligand-binding domain from a human progesterone receptor and a transactivation domain from the human RelA protein (p65). Burcin et al. (1999); Ye et al. (2002). Other exemplary SMR-activated transactivators than can be incorporated in an RCCHV include tetracycline/doxycycline-regulated tet-on repressors (Gossen & Bujard (1992) Proc Natl Acad Sci USA 89: 5547-51; Gossen et al. (1996) Science 268: 1766-69), and transactivators containing a ligand-binding domain of an insect ecdysone receptor (No et al. (1996) Proc Natl Acad Sci USA 93: 3346-51). A stringently ligand-dependent transactivator of this type is the RheoSwitch transactivator developed by Palli and colleagues (Palli et al. (2003) Eur J Biochem 270: 1308-15; Kumar et al. (2004) J Biol Chem 279: 27211-18). The RheoSwitch transactivator can be activated by ecdysteroids such as ponasterone A or muristerone A, or by synthetic diacylhydrazines such as RSL-1 (also known as RH-5849). Dhadialla et al. (1998) Annu Rev Entomol 43: 545-69. Other SMR-activated transactivators may be used, provided that they can be employed to stringently control the activity of a target gene and provided further that the associated SMRs have acceptably low toxicity in the hosts at their effective concentrations.

A concern has been whether pre-existing immunity to a virus will preclude its use as a vaccine or oncolytic vector. This issue not only relates to viruses such as adenoviruses and herpesviruses that are endemic but also to viruses that not normally infect humans but are used repeatedly as vectors. There may have been more serious concerns regarding the effects of pre-existing immunity to adenovirus (type 5) than to any other vector. Draper & Heeney (2010) Nat Rev Microbiol 8: 62-73. Steffensen et al. (2012) (PLoS ONE (2012) 7: e34884) demonstrated that pre-existing immunity does not interfere with the generation of memory CD8 T cells upon vaccination with a heterologous antigen-expressing modified Ad5 vector, providing a basis for an efficient recall response and protection against subsequent challenge. Furthermore, the transgene product-specific response could be boosted by re-vaccination. The issue of pre-existing immunity to herpesviruses has also been examined in multiple studies. Brockman & Knipe (2002) J Virol 76: 3678-87; Chahlavi et al. (1999) Gene Ther 6: 1751-58; Delman et al. (2000) Hum Gene Ther 11: 2465-72; Hocknell et al. (2002) J Virol 76: 5565-80; Lambright et al. (2000) Mol Ther 2: 387-93; Herrlinger et al. (1998) Gene Ther 5: 809-19; Lauterbach et al. (2005) J Gen Virol 86: 2401-10; Watanabe et al. (2007) Virology 357: 186-98. A majority of these studies reported little effect or only relatively minor effects on immune responses to herpesvirus-delivered heterologous antigens or even on anti-tumor efficacy of oncolytic herpesviruses. Brockman & Knipe (2002); Chahlavi et al. (1999); Delman et al. (2000); Hocknell et al. (2002); Lambright et al. (2000); Watanabe, D (2007). Only two studies reported substantial reductions of immune responses. Herrlinger (1998); Lauterbach et al. (2005). However, it appears that the results of these studies may not be generalized because compromised models were employed. One of the studies employed a tumor model that was only barely infectable with the mutant HSV strain used. Herrlinger (1998). The other study employed a chimeric mouse immune model in combination with a severely crippled HSV strain (ICP4-, ICP22-, ICP27-, vhs-) as the test vaccine. Lauterbach et al. (2005). All studies agreed that vaccine uses of herpesviruses are possible even in the presence of pre-existing immunity.

Herpesviruses have evolved a multitude of mechanisms for evading immune detection and avoiding destruction. Tortorella et al. (2000) Annu Rev Immunol 18: 861-926. Elimination or weakening of some of these mechanisms could further enhance the potency of an RCCHV. For example, HSV-1 and HSV-2 express protein ICP47. This protein binds to the cytoplasmic surfaces of both TAP1 and TAP2, the components of the transporter associated with antigen processing TAP. Advani & Roizman (2005) In: Modulation of Host Gene Expression and Innate Immunity by Viruses (ed. P. Palese), pp. 141-61, Springer Verlag. ICP47 specifically interferes with MHC class I loading by binding to the antigen-binding site of TAP, competitively inhibiting antigenic peptide binding. Virus-infected human cells (but much less so murine cells) are expected to be impaired in the presentation of antigenic peptides in the MHC class I context and, consequently, to also be resistant to killing by CD8+CTL. Deletion or disablement of the gene that encodes ICP47 ought to significantly increase the potency of a human RCCHV vaccine.

The potency of an RCCHV may also be enhanced by including in the viral genome an expressible gene for a cytokine or other component of the immune system. A vaccination study in mice in which replication-defective herpesvirus recombinants expressing various cytokines were compared demonstrated that virus-expressed IL-4 and IL-2 had adjuvant effects. Osiorio & Ghiasi (2003) J Virol 77: 5774-83. Further afield, modulation of dendritic cell function by GM-CSF was shown to enhance protective immunity induced by BCG and to overcome non-responsiveness to a hepatitis B vaccine. Nambiar et al. (2009) Eur J Immunol 40: 153-61; Chou et al. (2010) J Immunol 185: 5468-75.

Expanding upon the basic definition given on p. 9 above as it relates to vaccine uses, an effective amount of an RCCHV of the present disclosure is an amount that upon administration to a subject and induced limited replication therein results in a detectably enhanced functional immunity of the subject against the SARS CoV-2 strain from which the antigen expressed by the RCCHV was derived. It is expected that this immunity will be sufficiently broad to also protect against variant SARS CoV-2 strains and, perhaps, also against other coronaviruses. It is noted that a number of factors will influence what constitutes an effective amount of an RCCHV, including to some extent the site and route of administration of the virus to a subject as well as the precise activation regimen utilized. Effective amounts of an RCCHV will be determined in dose-finding experiments. Generally, an effective amount of an RCCHV of the present disclosure will be from about 103 to about 10¹⁰ plaque-forming units (pfu) of virus. More preferably, an effective amount will be from about 10⁴ to about 10⁹ pfu of virus, and even more preferably from about 10⁴ to about 10⁸ pfu of virus. Larger or smaller amounts may be indicated.

A composition of the present disclosure will comprise an effective amount of an RCCHV and, if an SMR is also administered as part of the composition, an effective amount of the SMR. It can be administered in the form of a fine powder, e.g., a lyophilizate, under certain circumstances (see, e.g., U.S. Pat. Appl. Publ. No 20080035143; Chen et al. (2017) J Control Release 255: 36-44). More conventionally, a composition of the present disclosure will be an aqueous composition comprising an RCCHV, a pharmaceutically acceptable carrier or excipient. and, as the case may be, an SMR. It may be administered parenterally to a subject as an aqueous solution or a fine powder, or, in the case of administration to a mucosal membrane (e.g., airways), possibly as an aerosol thereof. See, e.g., U.S. Pat. No. 5,952,220. The term parenteral as used herein includes intracutaneous (epidermis and/or dermis), subcutaneous, intravenous, intramuscular, intraarticular, intraarterial, intrasynovial, intrasternal, intrathecal, intralesional and intracranial injection or infusion techniques.

The compositions of the present disclosure will typically include a buffer component. The compositions will have a pH that is compatible with the intended use and is typically between about 6 and about 8. A variety of conventional buffers may be employed such as phosphate, citrate, histidine, Tris, Bis-Tris, bicarbonate and the like and mixtures thereof. In aqueous compositions (including in pre-lyophilizate solutions), the concentration of the buffer generally ranges from about 0.01 to about 0.25% w/v (weight/volume).

Compositions of the present disclosure comprising am RCCHV may further include, for example, preservatives, virus stabilizers, tonicity agents and/or viscosity-increasing substances. As mentioned before, they may also include an appropriate SMR, or a formulation comprising such SMR.

Preservatives used in parenteral products include phenol, benzyl alcohol, methyl paraben/propylparaben and phenoxyethanol. Phenoxyethanol appears to be the most widely used preservative found in vaccines. Preservatives are generally used in concentrations ranging from about 0.002 to about 1% w/v. Meyer (2007) J Pharm Sci 96: 3155-67. Preservatives may be present in compositions comprising an RCCHV at concentrations at which they do not interfere or only minimally interfere with the replication efficiency of the virus.

Osmolarity can be adjusted with tonicity agents to a value that is compatible with the intended use of the compositions. For example, the osmolarity may be adjusted to approximately the osmotic pressure of normal physiological fluids, which is approximately equivalent to about 0.9% w/v of sodium chloride in water. Examples of suitable tonicity-adjusting agents include, without limitation, chloride salts of sodium, potassium, calcium and magnesium, dextrose, glycerol, propylene glycol, mannitol, sorbitol and the like, and mixtures thereof. Preferably, in aqueous compositions, the tonicity agent(s) will be employed in an amount to provide a final osmotic value of 150 to 450 mOsm/kg, more preferably between about 220 to about 350 mOsm/kg and most preferably between about 270 to about 310 mOsm/kg.

If indicated, the aqueous compositions of the present disclosure can further include one or more viscosity-modifying agents such as cellulose polymers, including hydroxypropylmethyl cellulose, hydroxyethyl cellulose, ethylhydroxyethyl cellulose, hydroxypropyl cellulose, methyl cellulose, carboxymethyl cellulose, glycerol, carbomers, polyvinyl alcohol, polyvinyl pyrrolidone, alginates, carrageenans, guar, karaya, agarose, locust bean, tragacanth and xanthan gums. Such viscosity modifying components are typically employed in an amount effective to provide the desired degree of thickening. Viscosity-modifying agents may be present in compositions comprising an RCCHV at concentrations at which they do not interfere or only minimally interfere with the replication efficiency of the virus.

If the composition also contains an SMR, an effective amount of such SMR can be included in the composition. As also provided before, an effective amount of an SMR to be co-delivered with an effective amount of an RCCHV will be an amount that yields an effective concentration of the SMR in the inoculation site, which effective concentration enables at least one round of replication of the RCCHV in infected cells in that site. To maintain an SMR at an effective concentration for a more extended period, it may be included in the form of a slow-release formulation (see also below).

Methods for amplifying herpesviruses are well known in the laboratory art. Industrial scale-up has also been achieved. Hunter (1999) J Virol 73: 6319-26; Rampling et al. (2000) Gene Ther 7: 859-866; Mundle et al. (2013) PLoS ONE 8(2): e57224. Various methods for purifying viruses have been disclosed. See, e.g., Mundle et al. (2013) and references cited therein; Wolf and Reichl (2011) Expert Rev Vaccines 10: 1451-75.

While an SMR can be co-administered with an RCCHV in a single composition, a composition comprising an RCCHV and a composition comprising an SMR can also be administered separately. The latter composition will comprise an effective amount of an SMR, typically formulated together with one or more pharmaceutically acceptable carriers or excipients.

A composition comprising an SMR may be administered orally, parenterally, by inhalation spray, topically, rectally, nasally, buccally, vaginally or via an implanted reservoir, preferably by oral administration, administration by injection or deposition at the site of virus inoculation. The compositions may contain any conventional non-toxic, pharmaceutically acceptable carrier, adjuvant or vehicle. In some cases, the pH of the formulation may be adjusted with pharmaceutically acceptable acids, bases or buffers to enhance the stability of the formulated SMR or its delivery form.

Liquid dosage forms of an SMR for oral administration include pharmaceutically acceptable emulsions, microemulsions, solutions, suspensions, syrups and elixirs. In addition, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include, e.g., wetting agents, emulsifying and suspending agents, sweetening, flavoring, and perfuming agents.

Injectable preparations, for example sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic, parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

In order to prolong the effect of an SMR, it may be desirable to slow the absorption of the compound from, e.g., intracutaneous, subcutaneous or intramuscular injection. This may be accomplished by the use of a liquid suspension of crystalline or amorphous material with poor water solubility. The rate of absorption of the SMR then depends upon its rate of dissolution, which, in turn, may depend upon crystal size and crystalline form. Alternatively, delayed absorption of a parenterally administered SMR is accomplished by dissolving or suspending the compound in an oil vehicle. Injectable depot forms are made by forming microcapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration can be suppositories which can be prepared by mixing the SMR with suitable non-irritating excipients or carriers such as cocoa butter, polyethylene glycol or a suppository wax which are solid at ambient temperature but liquid at body temperature and therefore melt in the rectum or vaginal cavity and release the SMR.

Solid dosage forms for oral administration include capsules, tablets, pills, powders, and granules. In such solid dosage forms, the SMR is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution-retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the SMR only, or preferentially, in a certain part of the intestinal tract, optionally, in a delayed manner. Examples of embedding compositions that can be used include polymeric substances and waxes.

Dosage forms for topical, intradermal or transdermal administration of an SMR include ointments, pastes, creams, lotions, gels, powders, solutions, sprays, inhalants or patches. The SMR is admixed under sterile conditions with a pharmaceutically acceptable carrier and any preservatives or buffers as may be required.

The ointments, pastes, creams and gels may contain, in addition to an SMR, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the SMR, excipients such as lactose, talc, silicic acid, aluminum hydroxide, calcium silicates and polyamide powder, or mixtures of these substances. Sprays can additionally contain customary propellants.

Transdermal patches have the added advantage of providing controlled delivery of a compound. Such dosage forms can be made by dissolving or dispensing the compound in the proper medium. Absorption enhancers can also be used to increase the flux of the compound into or across the skin.

For pulmonary delivery, a composition comprising an effective amount of an SMR is formulated and administered to the subject in solid or liquid particulate form by direct administration e.g., inhalation into the respiratory system. Solid or liquid particulate forms of the SMR prepared for practicing the present disclosure include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. Nos. 5,767,068 and 5,508,269, and WO 98/43650). A discussion of pulmonary delivery of antibiotics is also found in U.S. Pat. No. 6,014,969.

What an effective amount of an SMR is will depend on the activity of the particular SMR employed, the route of administration, time of administration, the stability and rate of excretion of the particular SMR as well as the nature of the specific composition administered. It may also depend on the age, body weight, general health, sex and diet of the subject, other drugs used in combination or contemporaneously with the particular SMR employed and like factors well known in the medical arts.

Ultimately, what is an effective amount of an SMR can be determined in dose-finding experiments, in which replication of a heat- and SMR-controlled RCCHV in the inoculation site is assessed experimentally. Once an effective amount has been determined in animal experiments, it may be possible to estimate a human effective amount. “Guidance for Industry. Estimating the maximum safe starting dose for initial clinical trials for therapeutics in adult healthy volunteers”, U.S. FDA, Center for Drug Evaluation and Research, July 2005, Pharmacology and Toxicology. For example, as estimated from rat data, an effective human amount of orally administered mifepristone (for enabling at least one cycle of virus replication) will be between about 1 and about 100 μg/kg body weight.

Recitation of ranges of values herein are merely intended to serve as a shorthand method of referring individually to each separate value falling within the range, unless otherwise indicated herein, and each separate value is incorporated into the specification as if it were individually recited herein. The singular encompasses the plural, unless otherwise stated or clearly contradicted by context.

The description herein of any aspect or embodiment of the invention using terms such as reference to an element or elements is intended to provide support for a similar aspect or embodiment of the invention that “consists of′,” “consists essentially of” or “substantially comprises” that particular element or elements, unless otherwise stated or clearly contradicted by context (e. g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

The present disclosure will be understood more readily by reference to the following examples, which are provided by way of illustration and are not intended to be limiting in any way.

### Examples

The generation of the viral recombinants was performed by homologous recombination of engineered plasmids along with purified virion DNA into rabbit skin cells (RS) (or other cells in some Examples) by the calcium phosphate precipitation method as previously described. Bloom (1998) HSV (Methods Mol Med (1998) 10: 369-386). All recombination plasmids used to engineer the insertions of heterologous promoters and genes contained recombination sequences that were cloned from HSV-1 strain 17syn+.

### Example 1: Construction of Heat- and Antiprogestin-Controlled RCCHVs HSV-GS1 and HSV-GS3

Plasmid IN994 was created as follows: an HSV-1 upstream recombination arm was generated by amplification of HSV-1 DNA (from base pairs 95,441 to 96,090) with DB112 (5′-GAGCTC ATCACCGCAGGCGAGTCTCTT-3′) (SEQ ID NO: 1) and DB113 (5′-GAGCTCGGTCTTCGG GACTAATGCCTT-3′) (SEQ ID NO: 2). The product was digested with Sacl and inserted into the Sacl restriction site of pBluescript to create pUP. An HSV-1 downstream recombination arm was generated using primers DB115-Kpnl (5′-GGGGTACCGGTTTTGTTTTGTGTGAC-3′) (SEQ ID NO: 3) and DB120-Kpnl (5′-GGGGTACCGGTGTGTGATGATTTCGC-3′) (SEQ ID NO: 4) to amplify HSV-1 genomic DNA sequence between base pairs 96,092 and 96,538. The PCR product was digested with Kpnl, and cloned into Kpnl digested pUP to create pIN994, which recombines with HSV-1 at the intergenic UL43/44 region.

### HSV-GS1

HSV-GS1 contains a (SMR-activated) transactivator (TA) gene cassette inserted into the intergenic region between UL43 and UL44. In addition, the ICP4 promoter has been replaced with a GAL4-responsive promoter (GAL4-binding site-containing minimal promoter) in both copies of the short repeats. A first recombination plasmid pIN:TA1 was constructed by inserting a DNA segment containing a GLP65 (TA) gene under the control of a promoter cassette that combined a human HSP70B (heat shock) promoter and a GAL4-responsive promoter (described in Vilaboa et al. (2005)) into the multiple cloning site of plasmid pIN994, between flanking sequences of the HSV-1 UL43 and UL44 genes. The TA cassette was isolated from plasmid Hsp70/GAL4-GLP65 (Vilaboa et al. (2005)) and was cloned by 3-piece ligation to minimize the region that was amplified by PCR. For the left insert, Hsp70/GAL4-GLP65 was digested with BamHI and BstX1 and the resulting 2875 bp band was gel-purified. This fragment contains the Hsp70/GAL4 promoter cassette as well as the GAL4 DNA-binding domain, the progesterone receptor ligand-binding domain and part of the p65 activation domain of transactivator GLP65. The right insert was generated by amplifying a portion of pHsp70/GAL4-GLP65 with the primers TA.2803-2823.fwd (5′-TCGACAACTCCGAGTTTC AGC-3′) (SEQ ID NO: 5) and BGHpA.rev (5′-CTCCTCGCGGCCGCATCGATCCATAGAGCC CACCGCATCC-3′) (SEQ ID NO: 6). The 763 bp PCR product was digested with BstX1 and Notl, and the resultant 676 bp band was gel-purified. This band contained the 3′ end of the p65 activation domain and the BGHpA. For the vector, pIN994 was digested with BamHI and Notl, and the resulting 4099 bp fragment was gel-purified and shrimp alkaline phosphatase (SAP)-treated. The two inserts were then simultaneously ligated into the vector, creating an intact TA cassette. Subsequent to transformation, colony #14 was expanded, and the plasmid was verified by restriction enzyme analysis and then by sequence analysis.

One μg of pIN:TA1 was co-transfected with 2 μg of purified HSV-1 (syn17+) virion DNA into rabbit skin (RS) cells by calcium phosphate precipitation. The resulting pool of viruses was screened for recombinants by picking plaques, amplifying these plaques on 96 well plates of RS cells, and dot-blot hybridization with a ³²P-labeled DNA probe prepared by labeling a TA fragment by random-hexamer priming. A positive well was re-plaqued and re-probed 5 times and verified to contain the TA by PCR and sequence analysis. This intermediate recombinant was designated HSV-17GS43.

A second recombination plasmid, pBS-KS:GAL4-ICP4, was constructed that contained a GAL4-responsive promoter inserted in place of the native ICP4 promoter by cloning it in between the HSV-1 ICP4 recombination arms in the plasmid pBS-KS:ICP4Δpromoter. This placed the ICP4 transcript under the control of the heterologous GAL4 promoter. This particular promoter includes six copies of the yeast GAL4 UAS (upstream activating sequence), the adenovirus E1b TATA sequence and the synthetic intron Ivs8. This promoter was excised from the plasmid pGene/v5-HisA (Invitrogen Corp.) with Aatll and Hindlll, and the resulting 473 bp fragment was gel-purified. For the vector, pBS-KS:ICP4Δpromoter was digested with Aatll and Hindlll and the resulting 3962 bp fragment gel-purified and SAP-treated. Ligation of these two fragments placed the GAL4 promoter in front of the ICP4 transcriptional start-site. Subsequent to transformation, colony #5 was expanded, test-digested and verified by sequencing.

One μg of pBS-KS:GAL4-ICP4 was co-transfected with 4 μg of purified HSV-17GS43 virion DNA into cells of the ICP4-complementing cell line E5 (DeLuca and Schaffer (1987) Nucleic Acids Res 15: 4491-4511) by calcium phosphate precipitation. The resulting pool of viruses was screened for recombinants by picking plaques, amplifying these plaques on 96 well plates of E5 cells, and dot-blot hybridization with a ³²P-labeled DNA probe prepared by labeling the GAL4-responsive promoter fragment by random-hexamer priming. A strongly positive well was re-plaqued and re-probed 7 times and verified to contain the GAL4-responsive promoter in both copies of the short repeat sequences by PCR and sequence analysis. This recombinant was designated HSV-GS1.

To obtain pBS-KS:ΔSacl, the Sacl site was deleted from the polylinker of plasmid vector pBluescript-KS+, by digesting the plasmid with Sacl. The resulting 2954 bp fragment was gel-purified, treated with T4 DNA polymerase to produce blunt ends, re-circularized and self-ligated. Recombination plasmid BS-KS:ICP4Δpromoter was constructed as follows: to generate a first insert, cosmid COS48 (a gift of L. Feldman) was subjected to PCR with the primers HSV1.131428-131404 (5′-CTCCTCAAGCTTCTCGAGCACACGGAGCGCGGCTGC CGACAG-3′) (SEQ ID NO: 7) and HSV1.130859-130880 (5′-CTCCTCGGTACCCCATGG AGGCCAGCAGAGCCAGC-3′) (SEQ ID NO: 8). The primers placed Hindlll and Xhol sites on the 5′ end of the region, and Ncol and Kpnl sites on the 3′ end, respectively. The 600 bp primary PCR product was digested with Hindlll and Kpnl, and the resulting 587 bp fragment was gel-purified. Vector pBS-KS:ΔSacl was digested with Hindlll and Kpnl, and the resulting 2914 bp fragment was gel-purified and SAP-treated. Ligation placed the first insert into the vector's polylinker, creating pBS-KS:ICP4-3′ end. To generate a second insert, cosmid COS48 was subjected to PCR with the primers HSV1.132271-132250 (5′-CTCCTCGCGGCCGCA CTAGTTCCGCGTGTCCCTTTCCGATGC-3′) (SEQ ID NO: 9) and HSV1.131779-131800 (5′-CTCCTCCTCGAGAAGCTTATGCATGAGCTCGACGTCTCGGCGGTAATGAGATACGAGC-3′) (SEQ ID NO: 10). These primers placed Notl and Spel sites on the 5′ end of the region and Aatll, Sacl, Nsil, Hindlll and Xhol sites on the 3′ end, respectively. The 549 bp primary PCR product was digested with Notl and Xhol, and the resulting 530 bp band was gel-purified. This fragment also contained the 45 bp OriS hairpin. Plasmid BS-KS:ICP4-3′ end was digested with Notl and Xhol and the resulting 3446 bp band was gel-purified and SAP-treated. Ligation generated pBS-KS:ICP4Δpromoter. The inserts in pBS-KS:ICP4Δpromoter were verified by sequence analysis.

HSV-GS3 contains a (SMR-activated) transactivator (TA) gene cassette inserted into the intergenic region between UL43 and UL44. In addition, the ICP4 promoter has been replaced with a GAL4-responsive promoter (GAL4-binding site-containing minimal promoter) in both copies of the short repeats. Furthermore, the ICP8 promoter was replaced with a GAL4-responsive promoter. The construction of this recombinant virus involved placing a second HSV-1 replication-essential gene (ICP8) under control of a GAL4-responsive promoter. HSV-GS1 was used as the “backbone” for the construction of this recombinant. ICP8 recombination plasmid pBS-KS:GAL4-ICP8 was constructed. This plasmid contained a GAL4-responsive promoter inserted in place of the native ICP8 promoter by cloning it in between the HSV-1 ICP8 recombination arms in the plasmid pBS-KS:ICP8Δpromoter. This placed the ICP8 transcript under the control of the heterologous GAL4-responsive promoter. This GAL4-responsive promoter was excised from the plasmid pGene/v5-HisA (Invitrogen Corp.) with Aatll and Hindlll, and the resulting 473 bp fragment was gel-purified. For the vector, pBS-KS:ICP8Δpromoter was digested with Aatll and Hindlll, and the resulting 4588 bp fragment gel-purified and SAP-treated. Ligation of the latter two DNA fragments placed the GAL4-responsive promoter in front of the ICP8 transcriptional start-site. Subsequent to transformation, colony #10 was expanded, test-digested and verified by sequencing.

One μg of pBS-KS:GAL4-ICP8 was co-transfected with 10 μg of purified HSV-GS1 virion DNA into E5 cells by calcium phosphate precipitation. Subsequent to the addition of mifepristone to the medium, the transfected cells were exposed to 43.5° C. for 30 minutes and then incubated at 37° C. Subsequently, on days 2 and 3, the cells were again incubated at 43.5° C. for 30 minutes and then returned to 37° C. Plaques were picked and amplified on 96 well plates of E5 cells in media supplemented with mifepristone. The plates were incubated at 43.5° C. for 30 minutes 1 hour after infection and then incubated at 37° C. Subsequently, on days 2 and 3, the plates were also shifted to 43.5° C. for 30 minutes and then returned to 37° C. After the wells showed 90-100% CPE, the plates were dot-blotted and the dot-blot membrane hybridized with a ³²P-labeled DNA probe prepared by labeling the HSV-1 ICP8 promoter fragment that was deleted. A faintly positive well was re-plaqued and re-probed 8 times and verified to have lost the ICP8 promoter and to contain the GAL4-responsive promoter in its place by PCR and sequence analysis. This recombinant was designated HSV-GS3.

Recombination plasmid pBS-KS:ICP8Δpromoter was constructed using essentially the same strategy as that described above for the creation of pBS-KS:ICP4Δpromoter: a first insert was PCR-amplified from HSV-1 17syn+virion DNA using the primers HSV1.61841-61865 (5′-CTC CTCAGAACCCAGGACCAGGGCCACGTTGG-3′) (SEQ ID NO: 11) and HSV1.62053-62027 (5′-CTCCTCATGGAGACAAAGCCCAAGACGGCAACC-3′) (SEQ ID NO: 12) and subcloned to yield intermediate vector pBS-KS:ICP8-3′ end. A second insert was similarly obtained using primers HSV1.62173-62203 (5′-CTCCTCGGAGACCGGGGTTGGGGAATGAATCCCTCC-3′) (SEQ ID NO: 13) and HSV1.62395-62366 (5′-CTCCTCGCGGGGCGTGGGAGGGGCTGG GGCGGACC-3′) (SEQ ID NO: 14) and was subcloned into pBS-KS:ICP8-3′ end to yield pBS-KS:ICP8Δpromoter.

### Example 2: Construction of Heat- and Antiprogestin-Controlled RCCHV HSV-GS4 Expressing a Nonfunctional ICP47

HSV-GS4 contains a (SMR-activated) transactivator (TA) gene cassette inserted into the intergenic region between UL43 and UL44. In addition, the ICP4 promoter has been replaced with a GAL4-responsive promoter (GAL4-binding site-containing minimal promoter) in both copies of the short repeat, and the ICP8 promoter has been replaced with a GAL4-responsive promoter. Furthermore, the US12 gene has been mutated to render its protein product (ICP47) nonfunctional. ICP47 amino acid residue K31 was changed to G31, and R32 to G32. Neumann, L. et al. (1997) J Mol Biol 272: 484-92; Galocha, B. et al. (1997) J Exp Med 185: 1565-72. A 500 bp ICP47 coding sequence-containing fragment was PCR-amplified from virion DNA of strain 17syn+. The fragment was PCR-amplified as two pieces (a “left-hand” and a “right-hand” piece), using two primer pairs. The mutations were introduced through the 5′ PCR primer for the right-hand fragment. The resulting amplified left-hand and mutated right-hand fragments were subcloned into vector pBS, and the sequence in subclones was confirmed by sequence analysis. A subclone containing the 500 bp fragment with the desired mutations in ICP47 codons 31 and 32 was termed pBS:mut-ICP47.

One μg of pBS:mut-ICP47 was co-transfected with 10 μg of purified HSV-GS3 virion DNA into E5 cells by calcium phosphate precipitation. Subsequent to the addition of mifepristone to the medium, the transfected cells were exposed to 43.5° C. for 30 minutes and then incubated at 37° C. Subsequently on days 2 and 3, the cells were again incubated at 43.5° C. for 30 minutes and then returned to 37° C. Plaques were picked and amplified on 96 well plates of E5 cells in media supplemented with mifepristone. The plates were incubated at 43.5° C. for 30 minutes 1 hour after infection and then incubated at 37° C. Subsequently on days 2 and 3, the plates were also shifted to 43.5° C. for 30 minutes and then returned to 37° C. After the wells showed 90-100% CPE, the plates were dot-blotted and the dot-blot membrane hybridized with a ³²P-labeled oligonucleotide probe to the mutated ICP47 region. A positive well was re-plaqued and re-probed several times and verified by sequence analysis to contain the expected mutated ICP47 gene sequence. This recombinant was designated HSV-GS4.

Single step growth experiments indicated that, subsequent to activation (heat and mifepristone), recombinants HSV-GS1, HSV-GS3 and HSV-GS4 replicated essentially as well as the wildtype virus 17syn+ from which they were derived.

### Example 3: Construction of Heat- and Antiprogestin-Controlled RCCHV HSV-GS41 Expressing a Nonfunctional ICP47 and a Full-Length SARS CoV-2 Spike Protein

HSV-GS41 contains a transactivator (TA) gene cassette inserted into the intergenic region between UL43 and UL44. In addition, the ICP4 promoter has been replaced with a GAL4-responsive promoter (GAL4-binding site-containing minimal promoter) in both copies of the short repeats, and the ICP8 promoter has been replaced with a GAL4-responsive promoter. Furthermore, the US12 gene has been mutated to render its protein product (ICP47) nonfunctional. The recombinant further contains a spike protein gene from a SARS CoV-2 that is expressed from a CMV immediate early promoter. The recombination plasmid was constructed by the following sequential steps. First, the 814 bp fragment containing the region spanning the HSV-1 UL37/UL38 intergenic region from nt 83,603-84,417 from the plasmid NK470 was subcloned into pBS that had had the MCS removed (digestion with Kpnl/Sacl) to yield pBS:UL37/38. A cassette containing a synthetic CMV IE promoter flanked by the CpBS-SK+MCS was ligated into pBS:UL37/38 that was digested with BspE1/Aflll, which cuts between the UL37 and UL38 genes to yield the plasmid pIN:UL37/38. Plasmid “pCMV3-2019-nCoV Spike(S1+S2)-long” was obtained from Sino Biologicals (cat. No. VG40589-UT). The SARS CoV-2 spike gene sequence was excised from this plasmid and inserted behind the CMV promoter in the plasmid pIN:UL37/38 to yield plasmid pIN:37/38-S(SARS CoV-2). To produce recombinant HSV-GS41, RS cells were co-transfected with plasmid pIN:37/38-S(SARS CoV-2) and purified HSV-GS4 virion DNA. Subsequent to the addition of mifepristone to the medium, the co-transfected cells were exposed to 43.5° C. for 30 min and then incubated at 37° C. Subsequently, on days 2 and 3, the cells were again incubated at 43.5° C. for 30 min and then returned to 37° C. Picking and amplification of plaques, screening (using a ³²P-labeled DNA probe prepared from the spike protein-coding sequence) and plaque purification was performed essentially as described for HSV-GS3. The resulting plaque-purified recombinant HSV-GS41 was verified by Southern blot as well as by PCR and DNA sequence analysis of the recombination junctions.

### Example 4: Construction of Heat- and Antiprogestin-Controlled RCCHV HSV-GS31 Expressing a Full-Length SARS CoV-2 Spike Protein

To produce recombinant HSV-GS31, RS cells are co-transfected with plasmid pIN:37/38-S(SARS CoV-2) and purified HSV-GS3 virion DNA. Subsequent to the addition of mifepristone to the medium, the co-transfected cells are exposed to 43.5° C. for 30 min and then incubated at 37° C. Subsequently, on days 2 and 3, the cells are again incubated at 43.5° C. for 30 min and then returned to 37° C. Picking and amplification of plaques, screening (using a ³²P-labeled DNA probe prepared from the spike protein-coding sequence) and plaque purification are performed essentially as described for HSV-GS41. The resulting plaque-purified recombinant HSV-GS31 is verified by Southern blot as well as by PCR and DNA sequence analysis of the recombination junctions.

### Example 5: Isolation and Characterization of KRT1 and KRT77 Promoters

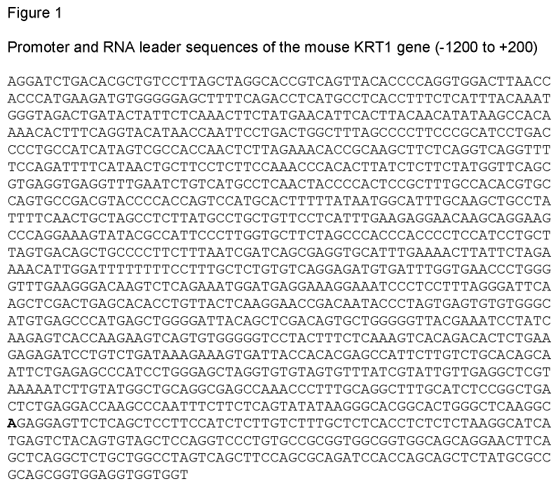

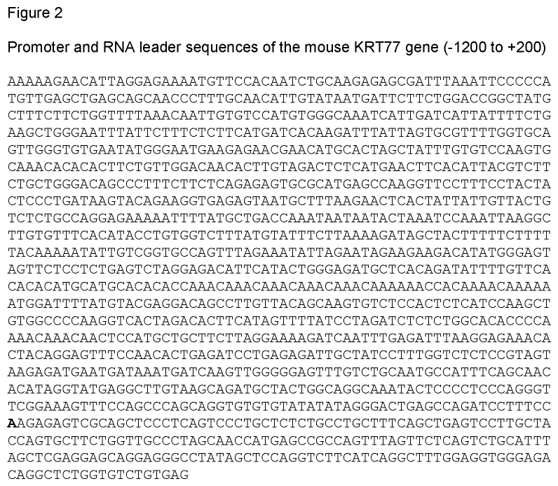

KRT1 and KRT77 promoter and RNA leader sequences were PCR-amplified from mouse genomic DNA using a standard protocol. Promoter sequences lie upstream from the start of transcription site (−), and RNA leader sequences begin at the start of transcription site (+). PCR amplification employed either primers mKRT1 F1 (5′-CTGACTGGCTTTAGCCCCTT-3′) (SEQ ID NO: 15) and mKRT1R1 (5′-GCCTTAGAGAGAGGTGAGAGC-3′) (SEQ ID NO: 16), or primers mKRT1F2 (5′-GCCACAAAACACTTTCAGGTACATA-3′) (SEQ ID NO: 17) and mKRT1R2 (5′-TGATGCCTTAGAGAGAGGTGA-3′) (SEQ ID NO: 18). For KRT77, the primers were mKRT77F1 (5′-AAGATTTATTAGTGCGTTTTGGTGC-3′) (SEQ ID NO: 19) and mKRT77R1 (5′-CAGAAGCACTGGTAGCAAGGA-3′) (SEQ ID NO: 20). The latter primer sequences were designed based on published mKRT1 and mKRT77 sequences (FIGS. 1 and 2) (SEQ ID NOS: 21 and 22). The amplified KRT1 and KRT77 segments were then subcloned into vector pGL4.16 (Promega Corp., Madison, WI) that contains a promoter-less luciferase reporter gene luc2CP from Photinus pyralis. To achieve this, the amplified KRT1 and KRT77 DNAs were further amplified with forward and reverse primers that also contained a Kpnl or a BamHI recognition sequence, respectively. The re-amplified DNAs were digested with Kpnl and BamHI, and the fragments were gel-purified and then ligated into Kpnl/Bglll-double-digested pGL4.16. Following transformation, colonies were picked and expanded, and inserts were subjected to nucleotide sequence analysis. Clone KRT1 1.2 contains mKRT1 sequences from position −993 to position+56, clone KRT1 2.3 mKRT1 sequences from position −1026 to position+60 and clone KRT77 3.2 mKRT77 sequences from position −986 to position+72.

The ability of the KRT promoters to drive transcription from the functionally linked luciferase reporter gene was assessed in several cell types. Since expression was to be compared between different cell types which may differ in transfectability and general transcriptional activity, the different KRT constructs were co-transfected with a construct that contained a β-galactosidase reporter gene controlled by the ubiquitously active ROSA promoter (pDRIVE-mROSA; InvivoGen Corp.). Transfection of subconfluent cultures grown under standard conditions employed a standard lipofectamine procedure. The activities of the KRT promoters were expressed as ratios of relative luciferase (LUC) to β-galactosidase (B-GAL) activities. LUC activity was measured using the Dual Glo Luciferase Assay System (Promega) and B-GAL activity using the Beta-Glo Assay System (Promega). Results from two representative experiments are shown in Tables 2 and 3. The experiment in Table 2 was conducted employing

TABLE 2
Activity of KRT promoters in Neuro2a cells

| | | Rel. LUC | | |
|---|---|---|---|---|
| | | activity − | Rel. | |
| | Rel. | pGL4.16 | B-GAL | LUC/ |
| | LUC | vector | activity | (B-GAL × |
| Construct | activity | background | (−background) | 10⁻⁴) |
| KRT1 1.2 | 70.5 +/− | 13.7 | 130073.7 +/− | 1.0 |
| | 7.3 | | 6589 | |
| KRT1 2.3 | 91.0 +/− | 34.2 | 136262.3 +/− | 2.5 |
| | 12.0 | | 3389 | |
| KRT77 3.2 | 55.3 +/− | 0 | 131378.7 +/− | 0 |
| | 29.6 | | 6127 | |
| pGL4.16 | 56.8 +/− | | 112490.0 +/− | |
| (promoter- | 36.8 | | 5862 | |
| less) | | | | |

TABLE 3
Activity of KRT promoters in HEK293T cells

| | | Rel. LUC | | | |
|---|---|---|---|---|---|
| | | activity - | | | |
| | | pGL4.16 | Rel. B-GAL | LUC/(B- | Selectivity |
| | Rel. LUC | vector | activity | GAL × | HEK293T/ |
| Construct | activity | background | (-background) | 10⁻⁴) | Neuro2a |
| KRT1 1.2 | 9500.9 +/− | 8400.8 | 36006.0 +/− | 2333.5 | 2333.5 |
| | 1029 | | 3995.9 | | |
| KRT1 2.3 | 6051.6 +/− | 4951.5 | 41178.5 +/− | 1201.8 | 480.7 |
| | 1486 | | 1267.1 | | |
| KRT77 3.2 | 20990 +/− | 19889.9 | 42632.5 +/− | 4668.9 | >4668.9 |
| | 2313 | | 3459.5 | | |
| pGL4.16 | 1100.1 +/− | | 36409.2 +/− | | |
| (promoter- | 172 | | 8205.9 | | |
| less) | | | | | |

mouse neural cell line Neuro2a, and the experiment in Table 3 with the human epithelial cell line HEK293T. The results demonstrate that the KRT1 and KRT77 promoters are highly active in the epithelial cells but practically inactive in the neural cells. Therefore, the isolated promoter segments contain all information required for their tissue-specific or selective activity.

Subsequently, a short and a long segment of the human KRT1 promoter were amplified from human genomic DNA. The PCR products were further amplified using primer pairs hKRT1 LongF Kpnl (5′-ATTAGGTACCGCAGCCGAAGGATTTTAGTGC-3′) (SEQ ID NO: 23) and hKRT1 R BamHI (5′-AGTAGGATCCGGAGCAAGGTAGAGTAAGGGAA-3′) (SEQ ID NO: 24) for the long segment, and hKRT1 ShortF Kpnl (5′-AGTCGGTACCTTCTATTGCTGGTG TCTGTCTC-3′) (SEQ ID NO: 25) and hKRT1 R BamHI (5′-AGTAGGATCCGGAGCAAGGT AGAGTAAGGGAA-3′) (SEQ ID NO: 24) for the short segment. The amplification products were double-digested with Kpnl and BamHI and ligated to Kpnl/Bglll-double digested vector pGL4.16. Following transformation, colonies were picked and expanded, and inserts were subjected to nucleotide sequence analysis. Clone hKRT1 L1 contains hKRT1 sequences from position −2001 to position+39 and clone hKRT1 S3 sequences from position −724 to position+39. Expression experiments similar to those described above were carried out using HEK293T epithelial cells and SHSY5Y human neural cells. The activities of hKRT1 L1 and hKRT1 S3 in epithelial cells and neural cells were comparable with those of mKRT1 1.2. In recombinants described below mouse KRT promoters were employed. Similar recombinants could be constructed using the latter shot or long hKRT1 promoters.

### Example 6: Heat- and Antiprogestin-Controlled RCCHV HSV-GS31-A Expressing a SARS CoV-2 Spike Protein

HSV-GS31-A contains a transactivator (TA) gene cassette inserted into the intergenic region between UL43 and UL44. In addition, the ICP4 promoter has been replaced with a GAL4-responsive promoter (GAL4-binding site-containing minimal promoter) in both copies of the short repeats, and the ICP8 promoter has been replaced with a mKRT1 promoter. The recombinant further contains a spike protein gene from a SARS CoV-2 that is expressed from a CMV immediate early promoter. Recombinant HSV-GS31-A is derived from HSV-GS19-A which is derived from HSV-GS19.

HSV-GS19 is derived from HSV-GS3 and contains an insertion between the UL37 and UL38 genes of a gene cassette expressing the A/California/07/2009 (H1N1) hemagglutinin (HA) gene driven by the CMV IE promoter.

The recombination plasmid was constructed by the following sequential steps. The complete coding sequence of the HA gene of A/California/07/2009 (Genbank accession no. KU933485) was synthesized de novo by GeneScript (Piscataway, NJ) and subcloned into pBluescript (pBS). The HA gene was excised from this plasmid and inserted behind the CMV promoter in the plasmid pIN:UL37/38 to yield plasmid pIN:37/38-California07//HA.

One μg of pIN:37/38-California07//HA was co-transfected with 10 μg of purified HSV-GS3 virion DNA into rabbit skin (RS) cells by calcium phosphate precipitation. Subsequent to the addition of mifepristone to the medium, the transfected cells were exposed to 43.5° C. for 30 minutes and then incubated at 37° C. Subsequently, on days 2 and 3, the cells were again incubated at 43.5° C. for 30 minutes and then returned to 37° C. Picking and amplification of plaques, screening (using a ³²P-labeled DNA probe prepared by labeling the HA gene) and plaque purification are performed essentially as described for HSV-GS3. One recombinant clone was verified by southern blot analysis as well as by sequencing of the HA insert and −200 bp of the flanking sequence on each side of the insert. This recombinant was designated HSV-GS19. Upon this genetic verification, a master stock was prepared.

HSV-GS19-A is derived from HSV-GS19. Its ICP8 gene is driven by a mKRT1 promoter.

Plasmid KRT1 1.2 containing the mKRT1 promoter was subjected to PCR amplification using primers mKRT1-Aatll F (5′-TAGTGACGTCCTGACTGGCTTTAGCCCCTT-3′) (SEQ ID NO: 26) and mKRT1-Aatll R (5′-AAAGACGTCGCCTTAGAGAGAGGTGAGAGCAAAGACA-3′) (SEQ ID NO: 27). The amplified fragment containing mKRT1 sequences from position −995 to position+55 was digested with Aatll and gel-purified. For the vector, pBS-KS:ICP8Δpromoter was digested with Aatll. The resulting 4.59 kbp fragment was gel-purified. Ligation of the latter two DNA fragments placed the mKRT1 promoter in front of the ICP8 transcriptional start-site. Subsequent to transformation, several colonies were expanded, and plasmid DNAs subjected to restriction and then sequence analysis to identify pBS-KS:mKRT1-ICP8. The correct orientation of the promoter was also verified by Hindlll digestion. The mKRT1 sequence contains a Hindlll site at position −917, and the polylinker of pBS-KS:ICP8Δpromoter also contains a Hindlll site.

To produce recombinant HSV-GS19-A, HEK293T cells were co-transfected with plasmid pBS-KS:mKRT1-ICP8 and purified HSV-GS19 virion DNA by calcium phosphate precipitation.

Subsequent to the addition of mifepristone to the medium, the transfected cells were exposed to 43.5° C. for 30 minutes and then incubated at 37° C. Subsequently, on days 2 and 3, the cells were again incubated at 43.5° C. for 30 minutes and then returned to 37° C. Plaques were picked and amplified on 96 well plates of HEK293T cells (in medium containing mifepristone). The plates were incubated at 43.5° C. for 30 minutes 1 hour after infection and then incubated at 37° C. Subsequently, on days 2 and 3, the were also shifted to 43.5° C. for 30 minutes and then returned to 37° C. After the wells showed 90-100% CPE, the plates were dot-blotted and the dot-blot membrane hybridized with a ³²P-labeled mKRT1 promoter DNA probe. A positive well was re-plaqued and re-probed several times, and was verified to have lost the GAL4 promoter at ICP8 and to contain the KRT1 promoter in its place by PCR and sequence analysis.

HSV-GS19-AA is derived from HSV-GS19. Its ICP8 gene is driven by an mKRT77 promoter.

To place the ICP8 gene under regulation of a mouse KRT77 promoter, plasmid pBS-KS:KRT77-ICP8 is constructed as follows. Plasmid KRT77 3.2 is subjected to PCR amplification using primers mKRT77AF1 (5′-GGACTGACGTCAAGATTTATTAGTGCGTTT TGGTGC-3′) (SEQ ID NO: 28) and mKRT77AR1 (5′-CAACCCGGGCAGAAGCACTG GTAGCAAGGA-3′) (SEQ ID NO: 29). The amplified fragment containing KRT77 sequences from position −986 to position+72 is digested with Aatll and Smal and is gel-purified. For the vector, plasmid pBS-KS:ICP8Δpromoter containing HSV-1 UL38 recombination arms is digested with Hindlll, ends are filled in using Klenow DNA polymerase, and the DNA is further digested with Aatll. The resulting 4.5-kbp fragment is gel-purified and SAP-treated. The latter two fragments are ligated. Following transformation, several colonies are amplified and tested for the presence of KRT77 sequences by restriction and sequence analysis. A clone containing the complete KRT77 promoter and RNA leader sequence of pKRT77 is designated pBS-KS:mKRT77-ICP8.

To produce recombinant HSV-GS19-AA, HEK293T cells are co-transfected with plasmid pBS-KS:mKRT77-ICP8 and purified HSV-GS19 virion DNA by calcium phosphate precipitation. Subsequent to the addition of mifepristone to the medium, the transfected cells are exposed to 43.5° C. for 30 minutes and then incubated at 37° C. Subsequently, on days 2 and 3, the cells are again incubated at 43.5° C. for 30 minutes and then returned to 37° C. Picking and amplification of plaques, screening (using a ³²P-labeled DNA probe prepared from the mKRT77 promoter sequence) and plaque purification are performed essentially as described for HSV-GS3. A positive well is verified to have lost the GAL4 promoter at ICP8 and to contain the mKRT77 promoter in its place by PCR and sequence analysis. HSV-GS19-AA could be employed instead of HSV-GS19-A in the constructions described below.

To produce recombinant HSV-GS31-A, HEK293T cells are co-transfected with plasmid pIN:37/38-S(SARS CoV-2) and purified HSV-GS19-A virion DNA. Subsequent to the addition of mifepristone to the medium, the co-transfected cells are exposed to 43.5° C. for 30 min and then incubated at 37° C. Subsequently, on days 2 and 3, the cells are again incubated at 43.5° C. for 30 min and then returned to 37° C. Picking and amplification of plaques, screening (using a ³²P-labeled DNA probe prepared from the spike protein-coding sequence) and plaque purification are performed essentially as described for HSV-GS3. The resulting plaque-purified recombinant HSV-GS31-A is verified by Southern blot as well as by PCR and DNA sequence analysis of the recombination junctions.

### Example 7: Construction of Heat-Controlled RCCHV HSV-GS31-B Expressing a SARS CoV-2 Spike Protein

HSV-GS31-B contains inserted human HSP70B promoters that replace the native promoters of the ICP4 genes. In addition, the ICP8 promoter has been replaced with a mKRT1 promoter. The recombinant further contains a spike protein gene from a SARS CoV-2 that is expressed from a CMV immediate early promoter. A transactivator (TA) gene cassette remains present in the intergenic region between UL43 and UL44 but is nonfunctional because of the absence of target genes. Recombinant HSV-GS31-B is derived from HSV-GS19-B.

HSV-GS19-B is derived from HSV-GS19-A. Its ICP4 genes are driven by human HSP70B promoters.

A short fragment of the human Hsp70B promoter (about 480 bp) was PCR-amplified using the primers Hsp70promAatllF (5′-ATTCGACGTCTCGCCTCAGGGATCCGACCT-3′) (SEQ ID NO: 30) and Hsp70promHindlllR (5′-TCTAGAGTCGACCTGCAGGCATGCAAGCTTCTTGT-3′) (SEQ ID NO: 31). The PCR fragment was digested with Aatll and Hind Ill and was ligated to Aatll/Hindll-digested pBS-KS:ICP4Δpromoter to generate recombination plasmid pHsp70Bpromoter-ICP4. To produce recombinant HSV-GS19-B, HEK293T cells were co-transfected with plasmid pHsp70Bpromoter-ICP4 and purified HSV-GS19-A virion DNA by calcium phosphate precipitation. The transfected cells were exposed to 43.5° C. for 30 minutes and then incubated at 37° C. Subsequently, on days 2 and 3, the cells were again incubated at 43.5° C. for 30 minutes and then returned to 37° C. Plaques were picked and amplified on 96 well plates of HEK293T cells. The plates were incubated at 43.5° C. for 30 minutes 1 hour after infection and then incubated at 37° C. Subsequently, on days 2 and 3, the were also shifted to 43.5° C. for 30 minutes and then returned to 37° C. After the wells showed 90-100% CPE, the plates were dot-blotted and the dot-blot membrane hybridized with a ³²P-labeled HSP70B promoter DNA probe. Several strongly positive wells were re-plaqued and re-probed several times, and one recombinant was verified to have lost the GAL4 promoter at ICP4 and to contain the HSP70B promoter in its place by PCR and sequence analysis.

To produce recombinant HSV-GS31-B, HEK293T cells are co-transfected with plasmid pIN:37/38-S(SARS CoV-2) and purified HSV-GS19-B virion DNA. The co-transfected cells are exposed to 43.5° C. for 30 min and then incubated at 37° C. Subsequently, on days 2 and 3, the cells are again incubated at 43.5° C. for 30 min and then returned to 37° C. Picking and amplification of plaques, screening (using a ³²P-labeled DNA probe prepared from the spike protein-coding sequence) and plaque purification are performed essentially as described for HSV-GS3. The resulting plaque-purified recombinant HSV-GS31-B is verified by Southern blot as well as by PCR and DNA sequence analysis of the recombination junctions.

### Example 8: Construction of Heat-Controlled RCCHV HSV-GS31-C Expressing a SARS CoV-2 Spike Protein

HSV-GS31-C contains a modified transactivator (TA) gene cassette inserted into the intergenic region between UL43 and UL44. In addition, the ICP8 promoter has been replaced with a mKRT1 promoter. The recombinant further contains a spike protein gene from a SARS CoV-2 that is expressed from a CMV immediate early promoter. Recombinant HSV-GS31-C is derived from HSV-GS19-C which is derived from HSV-GS19-A. The modified transactivator was derived from GLP65 (present in HSV-GS3) by the removal of the progesterone receptor ligand-binding domain. The gene for the modified transactivator is controlled by a human HSP70B promoter whose expression is limited by an 89 bp-long hairpin-forming sequence that has been inserted into the downstream RNA leader region.

Plasmid pINTAΔPRL-BD was prepared by removing the progesterone receptor ligand-binding domain in pIN:TA1 using the Q5 Site-Directed Mutagenesis Kit from New England Biolabs and primers DelGLP65 F (5′-GGGTCGACGCCCATGGAA-3′) (SEQ ID NO: 32) and DelGLP65 R (5′-CTGGTCGACACCCGGGAATTC-3) (SEQ ID NO: 33). To prepare pINTAΔPRL-BDΔGAL4, pINTAΔPRL-BD was digested with Sgfl, and the larger fragment generated was isolated and self-ligated. pINTAΔPRL-BDΔGAL4 is as pIN:TA1 except that the region encoding the progesterone receptor binding site in the transactivator gene as well as the GAL4 promoter were deleted. Plasmid pINTAΔPRL-BDΔGAL4Hairpin 4 was obtained by annealing de novo synthesized phosphorylated oligonucleotides Hairpin4F (5′-CCGGGATCCAACAACAACAA CAACCCTGCGGTCCACCACGGCCGATATCACGGCCGTGGTGGACCGCAGGGCAACAA CAACAACAACGGAT-3′) (SEQ ID NO: 34) and Hairpin4R (5′-CCGTTGTTGTTGTTGTTGCC CTGCGGTCCACCACGGCCGTGATATCGGCCGTGGTGGACCGCAGGGTTGTTGTTGTTG TTGGATCCCGGAT-3′) (SEQ ID NO: 35) and ligating the segment with Sgf-l-digested pINTAΔPRL-BDΔGAL4.

To produce recombinant HSV-GS19-C, HEK293T cells were co-transfected with plasmid pINTAΔPRL-BDΔGAL4Hairpin 4 and purified HSV-GS19-A virion DNA by calcium phosphate precipitation. The transfected cells were exposed to 43.5° C. for 30 minutes and then incubated at 37° C. Subsequently, on days 2 and 3, the cells were again incubated at 43.5° C. for 30 minutes and then returned to 37° C. Picking and amplification of plaques, screening (screening for the absence of the progesterone receptor sequence using a ³²P-labeled DNA probe prepared from the ligand-binding domain sequence of the GLP65 gene) and plaque purification were performed essentially as described for HSV-GS3. The resulting plaque-purified recombinant HSV-GS19-C was verified by Southern blot as well as by PCR and DNA sequence analysis of the recombination junctions.

To produce recombinant HSV-GS31-C, HEK293T cells are co-transfected with plasmid pIN:37/38-S(SARS CoV-2) and purified HSV-GS19-C virion DNA. The co-transfected cells are exposed to 43.5° C. for 30 min and then incubated at 37° C. Subsequently, on days 2 and 3, the cells are again incubated at 43.5° C. for 30 min and then returned to 37° C. Picking and amplification of plaques, screening (using a ³²P-labeled DNA probe prepared from the spike protein-coding sequence) and plaque purification are performed essentially as described for HSV-GS3. The resulting plaque-purified recombinant HSV-GS31-C is verified by Southern blot as well as by PCR and DNA sequence analysis of the recombination junctions.

### Example 9: Construction of Heat-Controlled RCCHV HSV-GS31-D Expressing a SARS CoV-2 Spike Protein

HSV-GS31-D contains a gene cassette for an expressible HSF1-derived constitutively active transactivator inserted into the intergenic region between UL43 and UL44. In addition, the ICP8 promoter has been replaced with a mKRT1 promoter. The recombinant further contains a spike protein gene from a SARS CoV-2 that is expressed from a CMV immediate early promoter. Recombinant HSV-GS31-D is derived from HSV-GS19-D which is derived from HSV-GS19-A.

Plasmid CMV-hHSF1 contains in between the Hindlll and EcoR1 sites of vector pcDNA3.1 a human HSF1 cDNA fragment that includes the entire 529 residues-long HSF1-coding sequence as well as upstream and downstream untranslated sequences (FIG. 3) (SEQ ID NO: 36). Baler et al. (1993) Mol Cell Biol 13: 2486-2496; Xia et al. (1999) Cell Stress Chaperon 4: 8-18. The single Notl site in pCMV-hHSF1 is destroyed by Notl digestion, filling-in using the Klenow fragment of DNA polymerase I, self-religation, transformation and isolation of a colony that lacks the Notl site. This plasmid is designated CMV-hHSF1-delNotl. A segment containing the sequence coding for hHSF1 residues 431-529 (encompassing the hHSF1 activation domain), 3′-nontranslated sequences of hHSF1 and the BGHpA region (present in the cDNA3.1 vector) is PCR-amplified from pCMV-hHSF1-delNotl using primers HSF1F (5′ GACGGTACCCCGACCTTGACAGCAGCCTG) (SEQ ID NO: 37) and BGHpA.rev (5′ CTCCTCGCGGCCGCATCGATCCATAGAGCCCACCGCATCC) (SEQ ID NO: 6). The amplified fragment is digested with Kpnl and Notl. Vector pSG424 (Sadowski and Ptasne (1989) Nucleic Acids Res 17: 7539) containing an expressible gene for a GAL4 DNA-binding domain (residues 1-147) is digested with Hindlll and Kpnl to release the GAL4(residues 1-147)-encoding fragment that is gel-purified. This fragment and the above Kpnl/Notl-digested PCR fragment from pCMV-hHSF1-delNotl are co-ligated into Hindlll/Not-double-digested and SAP-treated vector pBlueScript II SK. The resulting plasmid that contains the GAL4-HSF1 transactivator-coding sequence is designated pGAL4/HSF1TA. Plasmid GAL4/HSF1TA is digested with Hindlll and Notl, and the released transactivator-encoding fragment (1.48 kbp in length) is gel-purified. Construct p17 is digested with BamHI and Hindlll. A 0.45 kbp human HSP70B promoter fragment is gel-purified (Voellmy et al. (1985)). The latter two fragments are co-ligated with a pIN994 BamHI/Notl vector fragment. (pIN994 is digested with BamHI and Notl, and the resulting 4.10 kbp fragment is gel-purified and SAP-treated.) The resulting recombination plasmid is designated pIN: HSP70-TA.

Plasmid pIN: HSP70-TA is co-transfected with purified HSV-GS19-A virion DNA into HEK293T cells by calcium phosphate precipitation. The co-transfected cells are exposed to 43.5° C. for 30 min and then incubated at 37° C. Subsequently, on days 2 and 3, the cells are again incubated at 43.5° C. for 30 min and then returned to 37° C. Picking and amplification of plaques, (negative) screening (using a ³²P-labeled DNA probe prepared from the ligand-binding domain sequence of the GLP65 gene) and plaque purification are performed essentially as described for HSV-GS3. The resulting plaque-purified recombinant HSV-GS19-D is verified by Southern blot as well as by PCR and DNA sequence analysis of the recombination junctions.

To produce recombinant HSV-GS31-D, HEK293T cells are co-transfected with plasmid pIN:37/38-S(SARS CoV-2) and purified HSV-GS19-D virion DNA. The co-transfected cells are exposed to 43.5° C. for 30 min and then incubated at 37° C. Subsequently, on days 2 and 3, the cells are again incubated at 43.5° C. for 30 min and then returned to 37° C. Picking and amplification of plaques, screening (using a ³²P-labeled DNA probe prepared from the spike protein-coding sequence) and plaque purification are performed essentially as described for HSV-GS3. The resulting plaque-purified recombinant HSV-GS31-D is verified by Southern blot as well as by PCR and DNA sequence analysis of the recombination junctions.

All RCCHVs actually constructed were characterized for efficient, regulated replication in single step growth experiments in Vero cells or HEK293T cells, respectively. Expression of SARS CoV-2 spike protein was assessed by ELISA. Methods used were those described in Bloom et al. (2015) J Virol 89: 10668-79 and Bloom et al. (2018) J Virol 92: e00616-18.

### Example 10: Proof-of-Principle Experiments Relating to Tissue Restriction

Single-step growth experiments with HSV-GS19-A were carried out in different epithelial and neural cell lines essentially as described in Bloom et al. (2015). Parallel cultures of infected cells were subjected to heat treatment at 43.5° C. for 30 min in the presence of 10 nM ulipristal. At various times after heat treatment (up to 24 h), cultures were removed and virus titrated on E5 cells that had been previously transfected with an ICP8 expression construct. HSV-GS19-A was found to replicate efficiently in epithelial cells but not in neural cells.

Restricting ICP8 expression using an mKRT1 promoter also prevented reactivation from latency in the human Luhmes model (Edwards and Bloom (2019) J Virol 93: e02210-18). No reactivation was observed in human Luhmes cells infected with HSV-GS19-A even after activation with heat and ulipristal.

### Example 11: Induction of Immune Responses Against SARS-CoV-2 and Related Coronaviruses

Viruses HSV-GS41, HSV-GS31, HSV-GS31-A, HSV-GS31-B, HSV-GS31-C, HSV-GS31-D and HSV-GS4 (or HSV-GS3; negative virus control) or vehicle are administered under anesthesia to the plantar surfaces of both rear feet of adult female BALB/c mice (250,000 pfu virus per animal; 10 animals per group). Concurrently, and again 24 h later, the animals will receive an intraperitoneal injection of 50 μg/kg of body weight ulipristal (HSV-GS31, HSV-GS41and HSV-GS4 only). Three hours after inoculation, the mice are subjected to heat treatment (44.5° C. for 10 min) by immersion of their hind feet in a temperature-controlled water bath. Three weeks later, all animals will be reinoculated with 250,000 pfu/animal of the recombinant virus they had received before (or vehicle) as well as will be exposed to ulipristal as indicated and subjected to heat treatment as before. Three weeks later, mice from each group are anesthetized by inhalation of 2 to 3% isoflurane. The total blood volume of each mouse is collected, and the mice are euthanized by cervical dislocation. PBMCs are isolated by Ficoll gradient separation using Lymphoprep (Miltenyi Biotec, Bergisch Gladbach. Germany) according to the manufacturer's protocol. CoV-specific neutralizing antibody and CoV-specific responder cell frequency assays are carried out.

Pseudovirus-based neutralization assay: After collection, blood is allowed to clot for 30 min. After centrifugation at 800×g, the serum is collected. Serum samples are heated to 56° C. for 1 h to inactivate complement and are then diluted 1:10 in complete DMEM containing 10% heat-inactivated fetal bovine serum (FBS). VSV pseudoviruses presenting coronavirus spike proteins are prepared using the VSV pseudotyped virus (G*ΔG-VSV) as the backbone that carries in its genome an expression cassette for firefly luciferase instead of the VSV-G gene (Nie et al. (2020) Emerg Microbes Infect 9: 680-686). Briefly, HSK293T cells are transfected with expression plasmids for the spike proteins of SARS CoV-2, SARS CoV-1, MERS and HCoV-OC43 (all obtained from Sino Biologicals). Several hours later, the transfected cells are infected with G*ΔG-VSV. Pseudovirus is harvested about 24 h later and is titered on Vero cells, using luciferase activity as the readout. In the neutralization assay, the 50% inhibitor dilution (EC50) is defined as the serum dilution at which the relative light units measured by the luciferase activity assay are reduced by 50% compared with an appropriate control (virus+cells). Pseudovirus (about 650 TCID50/well) is incubated with serial dilutions of serum samples (six dilutions in a 3-fold step-wise manner) in duplicate for 1 h at 37° C., together with the virus control and cell control wells in hexaplicate. Then, freshly trypsinized Vero cells are added to each well (1.25−2.5×10⁴ cells/well). Following 24 h of incubation in a 5% CO₂ environment at 37° C., the luminescence is measured. The EC50 values are calculated with non-linear regression, i.e., log (inhibitor) vs. response (four parameters), using GraphPad Prism 8 (GraphPad Software, Inc., San Diego, CA, USA).

Responder cell frequency (RCF) assay: coronavirus-specific responder cells are quantified by a modified limiting dilution lymphoproliferation assay (Hayward et al. (1994) J Immunol Methods 170: 27-36), using recombinant spike proteins from SARS CoV-2, SARS-CoV-1 (SARS), MERS and HCoV-OC43 (obtained from Sino Biologicals) and control protein. Briefly, wells of 96-well plates are coated with 20 μl/well of antigens (spike proteins or control protein) and are allowed to air dry in a laminar flow hood. Dilutions of mouse PBMCs in DMEM are added to each well so that each well contains a minimum of 1 and a maximum of 10 lymphocytes per well in a volume of 100 μl complete medium (with serum). The plates are then incubated at 37° C. After 24 h, medium containing 10 μCi of [³H]thymidine is added to each plate for 12 h, the medium is replaced, and the plates are incubated for an additional 72 h. The wells are harvested, and the DNA is precipitated in 20 volumes of cold 10% trichloroacetic acid, transferred onto glass fiber discs (Whatman; GF/C) by filtration, rinsed with 95% ethanol, and dried using a heat lamp. The filters are then transferred to scintillation vials with Scintiverse (Fisher Scientific) and counted. The counts per minute (cpm) of [3H]thymidine are converted to RCF using the maximum-likelihood estimate method of Levin et al. (Levin et al. (2008) J Infect Dis 197: 825-835).

It is expected that HSV-GS41, HSV-GS31, HSV-GS31-A, HSV-GS31-B, HSV-GS31-C and HSV-GS31-D but not HSV-GS4 (or HSV-GS3) will be found to induce potent neutralizing antibody and cellular immune responses against SARS CoV-2. If results obtained with RCCV-based immunization with an influenza hemagglutinin (HSV-GS19) are an indication, cross-reactive immune responses may also be observed.

### Example 12: Vaccination Against Herpes Disease-an Immunization/Challenge Experiment

Previous immunization/challenge experiments demonstrated that immunization with replication-competent controlled viruses such as HSV-GS3 induced a strong protective response against challenge with wildtype herpesvirus. The following exemplary experiment is performed to demonstrate that RCCHVs of the present disclosure are similarly capable of protecting against disease caused by a subsequent herpesvirus infection. Induction of protective immunity is evaluated in a mouse footpad lethal challenge model (McKendall (1977) Infect Immun 16: 717-719). Viruses HSV-GS41, HSV-GS31, HSV-GS31-A, HSV-GS31-B, HSV-GS31-C, HSV-GS31-D, HSV-GS3 (or HSV-GS4) and KD6 (an ICP4- replication-incompetent HSV-1 recombinant (Dobson et al. 1990. Neuron 5:353-360) or vehicle are administered under anesthesia to the plantar surfaces of both rear feet of adult Swiss Webster outbred female mice (50,000 pfu per animal; 10 animals per group). Concurrently, and again 24 h later, all animals will receive an intraperitoneal injection of 50 μg/kg of body weight ulipristal (HSV-GS31, HSV-GS41and HSV-GS3 groups only). Three hours after inoculation, the mice are subjected to heat treatment (44.5° C. for 10 min) by immersion of their hind feet in a temperature-controlled water bath. Three weeks later, all animals will be reinoculated with 50,000 pfu/animal of the recombinant virus they had received before (or with vehicle for the mock group). The animals will be exposed to ulipristal as indicated and subjected to heat treatment as before. Three weeks later, all the animals are challenged with a 20-fold lethal dose of the HSV-1 wild-type strain 17syn+administered by the same route as the immunizing viruses. Survival of the animals is followed until no more lethal endpoints are reached, i.e., until all surviving animals have fully recovered. As is known from our previous experiments, replication-defective virus KD6 will induce a modest level of immunity. In contrast, activated (i.e., heat-treated in the presence of antiprogestin) HSV-GS3 will produce a complete or near complete protective effect. It is expected that HSV-GS41, HSV-GS31, HSV-GS31-A, HSV-GS31-B, HSV-GS31-C and HSV-GS31-D will produce comparable results as HSV-GS3. Hence, the latter RCCHVs employed to induce functional immunity against SARS CoV-2 and, possibly, other coronaviruses may also elicit or boost anti-herpetic immune responses.

Generally known molecular biology and biochemistry methods are/were used. Molecular biology methods are described, e.g., in “Current protocols in molecular biology”, Ausubel, F. M. et al., eds., John Wiley and Sons, Inc. ISBN: 978-0-471-50338-5.

1. A replication-competent controlled herpesvirus capable of delivering an antigen of a SARS CoV-2 virus, comprising inserted in the genome of an alpha-herpesvirus:
(a) a first exogenous promoter that is a nucleic acid sequence that acts as a heat shock promoter, the first promoter controlling the expression of a first replication-essential gene of the alpha-herpesvirus, and
(b) a second exogenous promoter and a functionally linked exogenous gene for an antigen of a SARS CoV-2 virus.

2. The replication-competent controlled herpesvirus of claim 1, further comprising a third exogenous promoter that is active in cells in a selected inoculation site of a mammalian subject to which site the replication-competent controlled herpesvirus is administered but is essentially inactive in cells of nerve ganglia of the mammalian subject, the third exogenous promoter being functionally linked to a second replication-essential gene of the replication-competent controlled herpesvirus.

3. The replication-competent controlled herpesvirus of claim 1, wherein the first exogenous promoter is functionally linked to an exogenous gene for a transactivator and the first replication-essential gene is functionally linked to a transactivator-responsive promoter.

4. The replication-competent controlled herpesvirus of claim 3, wherein the first exogenous promoter is a nucleic acid sequence that acts as a heat shock promoter as well as a transactivator-responsive promoter.

5. The replication-competent controlled herpesvirus of claim 3,
wherein the transactivator is a small-molecule regulator-activated transactivator.

6. The replication-competent controlled herpesvirus of claim 5, wherein the small-molecule regulator-activated transactivator contains a truncated ligand-binding domain from a progesterone receptor and is activated by an antiprogestin.

7. The replication-competent controlled herpesvirus of claim 1, further comprising an expressible exogenous gene for a repressor of the first replication-essential gene.

8. The replication-competent controlled herpesvirus of claim 1, wherein the replication-competent controlled virus is derived from a virus selected from the group consisting of an HSV-1, an HSV-2 and a varicella zoster virus.

9. The replication-competent controlled herpesvirus of claim 1, wherein the replication-competent controlled virus is derived from an HSV-1 or HSV-2 and is lacking a functional ICP47 gene.

10. A vaccine composition comprising an effective amount of the replication-competent controlled herpesvirus of claim 1.

11. Use of the replication-competent controlled herpesvirus of claim 1 for preventative or therapeutic vaccination against COVID-19 or another coronavirus-induced disease.

12. The replication-competent controlled herpesvirus of claim 2, wherein the first exogenous promoter is functionally linked to an exogenous gene for a transactivator and the first replication-essential gene is functionally linked to a transactivator-responsive promoter.

13. The replication-competent controlled herpesvirus of claim 12, wherein the first exogenous promoter is a nucleic acid sequence that acts as a heat shock promoter as well as a transactivator-responsive promoter.

14. The replication-competent controlled herpesvirus of claim 12, wherein the transactivator is a small-molecule regulator-activated transactivator.

15. The replication-competent controlled herpesvirus of claim 14, wherein the small-molecule regulator-activated transactivator contains a truncated ligand-binding domain from a progesterone receptor and is activated by an antiprogestin.
